SEQUENCE LISTING (64 sequences)

SEQ ID NO: 1 - protein, 5 aa, Homo sapiens
SGHRS

SEQ ID NO: 2 - protein, 6 aa, Homo sapiens
YFSETQ

SEQ ID NO: 3 - protein, 10 aa, Homo sapiens
ASRSDYNEQF

SEQ ID NO: 4 - protein, 110 aa, Homo sapiens
GVTQTPRYLIKTRGQQVTLSCSPISGHRSVSWYQQTPGQGLQFLFEYFSETQRNKGNFPGRFSGRQFSNSRSEMNVSTLELGDSALYLCASRSDYNEQFFGPGTRLTVLE

SEQ ID NO: 5 - protein, 6 aa, Homo sapiens
VSGLRG

SEQ ID NO: 6 - protein, 7 aa, Homo sapiens
LYSAGEE

SEQ ID NO: 7 - protein, 12 aa, Homo sapiens
AVQVLNAGNMLT

SEQ ID NO: 8 - protein, 112 aa, Homo sapiens
EDQVTQSPEALRLQEGESSSLNCSYTVSGLRGLFWYRQDPGKGPEFLFTLYSAGEEKEKERLKATLTKKESFLHITAPKPEDSATYLCAVQVLNAGNMLTFGGGTRLMVKPH

SEQ ID NO: 9 - protein, 309 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASRSDYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 10 - protein, 273 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQVLNAGNMLTFGGGTRLMVKPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 11 - DNA, 15 bp, Homo sapiens
  source 1..15: mol_type = genomic DNA
tctgggcataggagt

SEQ ID NO: 12 - DNA, 18 bp, Homo sapiens
  source 1..18: mol_type = genomic DNA
tacttcagtgagacacag

SEQ ID NO: 13 - DNA, 30 bp, Homo sapiens
  source 1..30: mol_type = genomic DNA
gccagcaggtcggattacaatgagcagttc

SEQ ID NO: 14 - DNA, 330 bp, Homo sapiens
  source 1..330: mol_type = genomic DNA
ggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcaggtcggattacaatgagcagttcttcgggccagggacacggctcaccgtgctagag

SEQ ID NO: 15 - DNA, 18 bp, Homo sapiens
  source 1..18: mol_type = genomic DNA
gtcagcggtttaagaggg

SEQ ID NO: 16 - DNA, 21 bp, Homo sapiens
  source 1..21: mol_type = genomic DNA
ctgtattcagctggggaagaa

SEQ ID NO: 17 - DNA, 36 bp, Homo sapiens
  source 1..36: mol_type = genomic DNA
gctgtgcaggtactgaatgcaggcaacatgctcacc

SEQ ID NO: 18 - DNA, 336 bp, Homo sapiens
  source 1..336: mol_type = genomic DNA
gaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcaggtactgaatgcaggcaacatgctcacctttggagggggaacaaggttaatggtcaaaccccat

SEQ ID NO: 19 - DNA, 927 bp, Homo sapiens
  source 1..927: mol_type = genomic DNA
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcaggtcggattacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggcgatggtcaagagaaaggattccagaggc

SEQ ID NO: 20 - DNA, 822 bp, Homo sapiens
  source 1..822: mol_type = genomic DNA
atggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcaggtactgaatgcaggcaacatgctcacctttggagggggaacaaggttaatggtcaaaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 21 - protein, 5 aa, Homo sapiens
LGHNT

SEQ ID NO: 22 - protein, 6 aa, Homo sapiens
FRNRAP

SEQ ID NO: 23 - protein, 11 aa, Homo sapiens
ASGTGQLSEQY

SEQ ID NO: 24 - protein, 112 aa, Homo sapiens
RVTQTPRHKVTEMGQEVTMRCQPILGHNTVFWYRQTMMQGLELLAYFRNRAPLDDSGMPKDRFSAEMPDATLATLKIQPSEPRDSAVYFCASGTGQLSEQYFGPGTRLTVTE

SEQ ID NO: 25 - protein, 6 aa, Homo sapiens
DSASNY

SEQ ID NO: 26 - protein, 7 aa, Homo sapiens
IRSNVGE

SEQ ID NO: 27 - protein, 9 aa, Homo sapiens
AAGGSSKII

SEQ ID NO: 28 - protein, 110 aa, Homo sapiens
ENVEQHPSTLSVQEGDSAVIKCTYSDSASNYFPWYKQELGKRPQLIIDIRSNVGEKKDQRIAVTLNKTAKHFSLHITETQPEDSAVYFCAAGGSSKIIFGSGTRLSIRPN

SEQ ID NO: 29 - protein, 311 aa, Homo sapiens
MATRLLCCVVLCLLGEELIDARVTQTPRHKVTEMGQEVTMRCQPILGHNTVFWYRQTMMQGLELLAYFRNRAPLDDSGMPKDRFSAEMPDATLATLKIQPSEPRDSAVYFCASGTGQLSEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 30 - protein, 270 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAAGGSSKIIFGSGTRLSIRPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - DNA, 15 bp, Homo sapiens
  source 1..15: mol_type = genomic DNA
ttaggccacaatact

SEQ ID NO: 32 - DNA, 18 bp, Homo sapiens
  source 1..18: mol_type = genomic DNA
ttccgcaaccgggctcct

SEQ ID NO: 33 - DNA, 33 bp, Homo sapiens
  source 1..33: mol_type = genomic DNA
gctagtggcacaggacagttaagcgagcagtac

SEQ ID NO: 34 - DNA, 336 bp, Homo sapiens
  source 1..336: mol_type = genomic DNA
agagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtatttttgtgctagtggcacaggacagttaagcgagcagtacttcgggccgggcaccaggctcacggtcacagag

SEQ ID NO: 35 - DNA, 18 bp, Homo sapiens
  source 1..18: mol_type = genomic DNA
gacagtgcctcaaactac

SEQ ID NO: 36 - DNA, 21 bp, Homo sapiens
  source 1..21: mol_type = genomic DNA
attcgttcaaatgtgggcgaa

SEQ ID NO: 37 - DNA, 27 bp, Homo sapiens
  source 1..27: mol_type = genomic DNA
gcagcagggggaagttccaagataatc

SEQ ID NO: 38 - DNA, 330 bp, Homo sapiens
  source 1..330: mol_type = genomic DNA
gagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcagggggaagttccaagataatctttggatcagggaccagactcagcatccggccaaat

SEQ ID NO: 39 - DNA, 933 bp, Homo sapiens
  source 1..933: mol_type = genomic DNA
atggccaccaggctcctctgctgtgtggttctttgtctcctgggagaagagcttatagatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtatttttgtgctagtggcacaggacagttaagcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggcgatggtcaagagaaaggattccagaggc

SEQ ID NO: 40 - DNA, 813 bp, Homo sapiens
  source 1..813: mol_type = genomic DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcagggggaagttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 41 - protein, 5 aa, Homo sapiens
SEHNR

SEQ ID NO: 42 - protein, 6 aa, Homo sapiens
FQNEAQ

SEQ ID NO: 43 - protein, 13 aa, Homo sapiens
ASSLGGRSQETQY

SEQ ID NO: 44 - protein, 114 aa, Homo sapiens
GVSQDPRHKITKRGQNVTFRCDPISEHNRLYWYRQTLGQGPEFLTYFQNEAQLEKSRLLSDRFSAERPKGSFSTLEIQRTEQGDSAMYLCASSLGGRSQETQYFGPGTRLLVLE

SEQ ID NO: 45 - protein, 7 aa, Homo sapiens
TRDTTYY

SEQ ID NO: 46 - protein, 8 aa, Homo sapiens
RNSFDEQN

SEQ ID NO: 47 - protein, 14 aa, Homo sapiens
ALRPNYGGSQGNLI

SEQ ID NO: 48 - protein, 117 aa, Homo sapiens
QKVTQAQTEISVVEKEDVTLDCVYETRDTTYYLFWYKQPPSGELVFLIRRNSFDEQNEISGRYSWNFQKSTSSFNFTITASQVVDSAVYFCALRPNYGGSQGNLIFGKGTKLSVKPN

SEQ ID NO: 49 - protein, 313 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLGGRSQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 50 - protein, 278 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALRPNYGGSQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 51 - DNA, 15 bp, Homo sapiens
  source 1..15: mol_type = genomic DNA
tctgaacacaaccgc

SEQ ID NO: 52 - DNA, 18 bp, Homo sapiens
  source 1..18: mol_type = genomic DNA
ttccagaatgaagctcaa

SEQ ID NO: 53 - DNA, 39 bp, Homo sapiens
  source 1..39: mol_type = genomic DNA
gccagcagcttaggagggagatcccaagagacccagtac

SEQ ID NO: 54 - DNA, 342 bp, Homo sapiens
  source 1..342: mol_type = genomic DNA
ggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttaggagggagatcccaagagacccagtacttcgggccaggcacgcggctcctggtgctcgag

SEQ ID NO: 55 - DNA, 21 bp, Homo sapiens
  source 1..21: mol_type = genomic DNA
acccgtgatactacttattac

SEQ ID NO: 56 - DNA, 24 bp, Homo sapiens
  source 1..24: mol_type = genomic DNA
cggaactcttttgatgagcaaaat

SEQ ID NO: 57 - DNA, 42 bp, Homo sapiens
  source 1..42: mol_type = genomic DNA
gctctgagacctaattatggaggaagccaaggaaatctcatc

SEQ ID NO: 58 - DNA, 351 bp, Homo sapiens
  source 1..351: mol_type = genomic DNA
cagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagacctaattatggaggaagccaaggaaatctcatctttggaaaaggcactaaactctctgttaaaccaaat

SEQ ID NO: 59 - DNA, 939 bp, Homo sapiens
  source 1..939: mol_type = genomic DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttaggagggagatcccaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggcgatggtcaagagaaaggattccagaggc

SEQ ID NO: 60 - DNA, 837 bp, Homo sapiens
  source 1..837: mol_type = genomic DNA
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagacctaattatggaggaagccaaggaaatctcatctttggaaaaggcactaaactctctgttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 61 - protein, 27 aa, Homo sapiens
RAKRSGSGATNFSLLKQAGDVEENPGP

SEQ ID NO: 62 - DNA, 81 bp, Homo sapiens
  source 1..81: mol_type = genomic DNA
cgtgccaagcgaagcggatctggcgccacgaacttctctctgttaaagcaagcaggagatgttgaagaaaaccccgggcct

SEQ ID NO: 63 - protein, 763 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKPLDPGSPQLPEHLESQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 64 - protein, 9 aa, synthetic construct
IYTWIEDHF

(19) United States
(12) Patent Application Publication
(10) Pub. No.: US 2026/0297186 A1
(43) Pub. Date: Oct. 1, 2026

(54) T-CELL RECEPTORS TARGETING FOXM1 AND METHODS OF USE THEREOF

(71) Applicant: Board of Regents, The University of Texas System, Austin, TX (US)

(72) Inventors: Alexandre Reuben, Houston, TX (US); Minying Zhang, Houston, TX (US); John Victor Heymach, Houston, TX (US); Gregory A. Lizee, Houston, TX (US); Peixin Jiang, Houston, TX (US); Patrick Hwu, Houston, TX (US)

(73) Assignee: Board of Regents, The University of Texas System, Austin, TX (US)

(21) Appl. No.: 19/480,700

(22) PCT Filed: Jul. 1, 2024
(86) PCT No.: PCT/US2024/036413

§ 371 Date: Oct. 31, 2025

Related U.S. Application Data
(60) Provisional application No. 63/512,267, filed on Jul. 6, 2023.

(51) Int. Cl. C07K 16/28; C07K 14/705

(52) U.S. Cl. CPC C07K 16/28; C07K 14/7051; C07K 2317/31

(57) ABSTRACT

The present disclosure provides T-cell receptors comprising an antigen binding variable region that specifically binds an antigenic peptide derived from FOXM1. The present disclosure further provides activated or engineered T-cells comprising the T-cell receptors of the disclosure, and methods of making and using these T-cell receptors. Aspects of the disclosure further relate to a polypeptide comprising an antigen binding variable region comprising a CDR3 region of the disclosure.

### CROSS-REFERENCE TO RELATED APPLICATIONS

This application claims the priority of U.S. Provisional Appl. Ser. No. 63/512,267, filed Jul. 6, 2023, the entire disclosure of which is incorporated herein by reference.

### INCORPORATION OF SEQUENCE LISTING

A sequence listing containing the file named “MDCC011WO_ST26.xml” which is 65.0 kilobytes (measured in MS-Windows®) and created on Jun. 17, 2024, and comprises 64 sequences, is incorporated herein by reference in its entirety.

### FIELD OF THE INVENTION

The present disclosure relates to the field of cancer immunotherapy, and more specifically to compositions and methods for the treatment of cancers that express or overexpress the protein FOXM1.

### BACKGROUND OF THE INVENTION

The FOXM1 gene encodes the transcription factor forkhead box protein M1(FOXM1), which is associated with the processes of cell proliferation, self-renewal, and tumorigenesis. FOXM1 is often amplified or overexpressed in tumors of varying histology. Several splice variants of FOXM1 may be expressed in cells, non-limiting examples of which include FOXM1A, FOXM1B, FOXM1C, and FOXM1D.

Adoptive T-cell therapy (ACT) refers to the practice of administering tumor-specific T-cells to a patient with cancer. The goal of adoptive T-cell therapy is that the transferred cells will recognize and kill the cancer cells without causing significant damage to healthy tissues. Adoptive T-cell therapy may utilize, for example, T-cells isolated from the patient or a donor and expanded in the laboratory prior to transfer. Adoptive T-cell therapy may also utilize genetically modified T-cells which express, for example, chimeric antigen receptors (CARs) or T-cell receptors (TCRs). Adoptive T-cell therapy using CARs and TCRs has demonstrated impressive results in several clinical trials. The efficacy of adoptive T-cell therapy is determined, at least in part, by the TCRs' interaction with peptide-major histocompatibility complexes (pMHCs), which comprise a peptide bound to an MHC molecule.

Intracellular antigenic proteins are cleaved into peptide chains within the cell, and these antigenic peptide chains then bind MHC molecules to form pMHCs, which are displayed on the extracellular surface. Cytoplasmic proteins are generally cleaved into peptide chains by proteolysis, and then form pMHCs with class I MHC molecules. Class I MHC molecules are expressed on the extracellular surface of almost all nucleated cells. In contrast to class I MHC molecules, class II MHC molecules are only expressed on the surface of certain cells, these cells are known as antigen-presenting cells (APCs). Antigen-presenting cells internalize extracellular proteins, for example by phagocytosis or endocytosis, cleave these proteins into peptide chains, and then form pMHCs with class II MHC molecules. TCRs recognize both class I and class II pMHCs.

TCRs for use in adoptive T-cell therapy must be matched to a patient's human leukocyte antigen (HLA) allele. The human class I MHC protein and the human class II MHC protein are each encoded by 3 gene regions. Human class I MHC is encoded by HLA-A, HLA-B, and HLA-C, and human class II MHC is encoded by HLA-DR, HLA-DP, and HLA-DQ. There is a continuing need in the art for novel cancer specific TCRs for use in cancer treatment.

### SUMMARY OF THE INVENTION

In one aspect, the present disclosure provides a recombinant polynucleotide molecule comprising a nucleotide sequence that encodes an antigen binding variable region comprising a CDR3 region, wherein said CDR3 region comprises an amino acid sequence selected from the group consisting of SEQ ID NO:3, SEQ ID NO:23, and SEQ ID NO:43. In one embodiment, the CDR3 region comprises the amino acid sequence of SEQ ID NO:3, and the antigen binding variable region further comprises a CDR1 region comprising the amino acid sequence of SEQ ID NO:1. In another embodiment, the CDR3 region comprises the amino acid sequence of SEQ ID NO:3, and the antigen binding variable region further comprises a CDR2 region comprising the amino acid sequence of SEQ ID NO:2. In yet another embodiment, the CDR3 region comprises the amino acid sequence of SEQ ID NO:23, and the antigen binding variable region further comprises a CDR1 region comprising the amino acid sequence of SEQ ID NO:21. In still yet another embodiment, the CDR3 region comprises the amino acid sequence of SEQ ID NO:23, and the antigen binding variable region further comprises a CDR2 region comprising the amino acid sequence of SEQ ID NO:22. The CDR3 region, in one embodiment, comprises the amino acid sequence of SEQ ID NO:43, and the antigen binding variable region further comprises a CDR1 region comprising the amino acid sequence of SEQ ID NO:41. The CDR3 region, in another embodiment, comprises the amino acid sequence of SEQ ID NO:43, and the antigen binding variable region further comprises a CDR2 region comprising the amino acid sequence of SEQ ID NO:42. The CDR3 region, in yet another embodiment, comprises the amino acid sequence of SEQ ID NO:3, and the antigen binding variable region further comprises an amino acid sequence having at least about 85% sequence identity to the amino acid sequence of SEQ ID NO:4. The CDR3 region, in still yet another embodiment, comprises the amino acid sequence of SEQ ID NO:23, and the antigen binding variable region further comprises an amino acid sequence having at least about 85% sequence identity to the amino acid sequence of SEQ ID NO:24. In one embodiment, the CDR3 region comprises the amino acid sequence of SEQ ID NO:43, and the antigen binding variable region further comprises an amino acid sequence having at least about 85% sequence identity to the amino acid sequence of SEQ ID NO:44.

In another aspect, the present disclosure provides a recombinant polynucleotide molecule comprising a nucleotide sequence that encodes an antigen binding variable region comprising a CDR3 region, wherein said CDR3 region comprises an amino acid sequence selected from the group consisting of SEQ ID NO:7, SEQ ID NO:27, and SEQ ID NO:47. In one embodiment, the CDR3 region comprises the amino acid sequence of SEQ ID NO:7, and the antigen binding variable region further comprises a CDR1 region comprising the amino acid sequence of SEQ ID NO:5. In another embodiment, the CDR3 region comprises the amino acid sequence of SEQ ID NO:7, and the antigen binding variable region further comprises a CDR2 region comprising the amino acid sequence of SEQ ID NO:6. In yet another embodiment, the CDR3 region comprises the amino acid sequence of SEQ ID NO:27, and the antigen binding variable region further comprises a CDR1 region comprising the amino acid sequence of SEQ ID NO:25. In still yet another embodiment, the CDR3 region comprises the amino acid sequence of SEQ ID NO:27, and the antigen binding variable region further comprises a CDR2 region comprising the amino acid sequence of SEQ ID NO:26. The CDR3 region, in one embodiment, comprises the amino acid sequence of SEQ ID NO:47, and the antigen binding variable region further comprises a CDR1 region comprising the amino acid sequence of SEQ ID NO:45. The CDR3 region, in another embodiment, comprises the amino acid sequence of SEQ ID NO:47, and the antigen binding variable region further comprises a CDR2 region comprising the amino acid sequence of SEQ ID NO:46. The CDR3 region, in yet another embodiment, comprises the amino acid sequence of SEQ ID NO:7, and the antigen binding variable region further comprises an amino acid sequence having at least about 85% sequence identity to the amino acid sequence of SEQ ID NO:8. The CDR3 region, in still yet another embodiment, comprises the amino acid sequence of SEQ ID NO:27, and the antigen binding variable region further comprises an amino acid sequence having at least about 85% sequence identity to the amino acid sequence of SEQ ID NO:28. In one embodiment, the CDR3 region comprises the amino acid sequence of SEQ ID NO:47, and the antigen binding variable region further comprises an amino acid sequence having at least about 85% sequence identity to the amino acid sequence of SEQ ID NO:48.

In certain embodiments, the present disclosure provides a polypeptide encoded by a recombinant polynucleotide molecule of the present disclosure. In one embodiment, a polypeptide of the present disclosure may comprise or be encoded by any one of SEQ ID NOs: 1-62 or may comprise or be encoded by a sequence having at least about 80% sequence identity to any one of SEQ ID NOs: 1-62, or a fragment thereof. In another embodiment, a polypeptide of the present disclosure may specifically bind an antigenic peptide derived from FOXM1. The present disclosure further provides, in particular embodiments, a composition comprising a polypeptide of the present disclosure. In one embodiment, a cell comprising a recombinant polynucleotide molecule of the present disclosure is provided. In another embodiment, the present disclosure provides a chimeric antigen receptor comprising a polypeptide of the present disclosure. In yet another embodiment, a polynucleotide molecule of the present disclosure may comprise or encode any one of SEQ ID NOs: 1-62 or may comprise or encode a sequence having at least 80% sequence identity to any one of SEQ ID NOs: 1-62, or a fragment thereof. In still yet another embodiment, the present disclosure provides a method of producing an engineered cell, the method comprising introducing a recombinant polynucleotide molecule of the present disclosure into a cell.

In yet another aspect, the present disclosure provides a method of treating cancer, the method comprising administering a polypeptide or a cell of the present disclosure to a subject in need thereof. In one embodiment, the cancer comprises a cancer cell expressing a FOXM1 protein presented on HLA-A*24:02. In another embodiment, the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

In still yet another aspect, the present disclosure provides a composition comprising a polypeptide, wherein said polypeptide comprises an antigen binding variable region comprising a CDR3 region, wherein said CDR3 region comprises an amino acid sequence selected from the group consisting of SEQ ID NO:3, SEQ ID NO:7, SEQ ID NO:23, SEQ ID NO: 27, SEQ ID NO:43, and SEQ ID NO:47. In one embodiment, the CDR3 region comprises the amino acid sequence of SEQ ID NO:3, and the antigen binding variable region further comprises a CDR1 region comprising the amino acid sequence of SEQ ID NO:1. In another embodiment, the CDR3 region comprises the amino acid sequence of SEQ ID NO:3, and the antigen binding variable region further comprises a CDR2 region comprising the amino acid sequence of SEQ ID NO:2. In yet another embodiment, the CDR3 region comprises the amino acid sequence of SEQ ID NO:7, and the antigen binding variable region further comprises a CDR1 region comprising the amino acid sequence of SEQ ID NO:5. In still yet another embodiment, the CDR3 region comprises the amino acid sequence of SEQ ID NO:7, and the antigen binding variable region further comprises a CDR2 region comprising the amino acid sequence of SEQ ID NO:6. The CDR3 region comprises, in one embodiment, the amino acid sequence of SEQ ID NO:23, and the antigen binding variable region further comprises a CDR1 region comprising the amino acid sequence of SEQ ID NO:21. The CDR3 region comprises, in another embodiment, the amino acid sequence of SEQ ID NO:23, and the antigen binding variable region further comprises a CDR2 region comprising the amino acid sequence of SEQ ID NO: 22. The CDR3 region comprises, in yet another embodiment, the amino acid sequence of SEQ ID NO:27, and the antigen binding variable region further comprises a CDR1 region comprising the amino acid sequence of SEQ ID NO:25. The CDR3 region comprises, in still yet another embodiment, the amino acid sequence of SEQ ID NO:27, and the antigen binding variable region further comprises a CDR2 region comprising the amino acid sequence of SEQ ID NO: 26. In one embodiment, the CDR3 region comprises the amino acid sequence of SEQ ID NO: 43, and the antigen binding variable region further comprises a CDR1 region comprising the amino acid sequence of SEQ ID NO:41. In another embodiment, the CDR3 region comprises the amino acid sequence of SEQ ID NO:43, and the antigen binding variable region further comprises a CDR2 region comprising the amino acid sequence of SEQ ID NO: 42. In yet another embodiment, the CDR3 region comprises the amino acid sequence of SEQ ID NO:47, and the antigen binding variable region further comprises a CDR1 region comprising the amino acid sequence of SEQ ID NO:45. In still yet another embodiment, the CDR3 region comprises the amino acid sequence of SEQ ID NO:47, and the antigen binding variable region further comprises a CDR2 region comprising the amino acid sequence of SEQ ID NO: 46. The CDR3 region comprises, in one embodiment, the amino acid sequence of SEQ ID NO: 3, and the antigen binding variable region further comprise an amino acid sequence having at least about 85% sequence identity to the amino acid sequence of SEQ ID NO:4. The CDR3 region comprises, in another embodiment, the amino acid sequence of SEQ ID NO:7, and the antigen binding variable region further comprise an amino acid sequence having at least about 85% sequence identity to the amino acid sequence of SEQ ID NO:8. The CDR3 region comprises, in yet another embodiment, the amino acid sequence of SEQ ID NO:23, and the antigen binding variable region further comprise an amino acid sequence having at least about 85% sequence identity to the amino acid sequence of SEQ ID NO:24. The CDR3 region comprises, in still yet another embodiment, the amino acid sequence of SEQ ID NO:27, and the antigen binding variable region further comprise an amino acid sequence having at least about 85% sequence identity to the amino acid sequence of SEQ ID NO:28. In one embodiment, the CDR3 region comprises the amino acid sequence of SEQ ID NO:43, and the antigen binding variable region further comprise an amino acid sequence having at least about 85% sequence identity to the amino acid sequence of SEQ ID NO:44. In another embodiment, the CDR3 region comprises the amino acid sequence of SEQ ID NO:47, and the antigen binding variable region further comprise an amino acid sequence having at least about 85% sequence identity to the amino acid sequence of SEQ ID NO:48. In yet another embodiment, the composition is serum-free, endotoxin-free, or sterile. In still yet another embodiment, the present disclosure provides a method of treating cancer, the method comprising administering a composition of the present to a subject in need thereof. The cancer, in one embodiment, comprises a cancer cell expressing a FOXM1 protein presented on HLA-A*24:02. Non-limiting examples of cancer cells that may be treated according to certain embodiments of the present disclosure include a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

In one aspect, the present disclosure provides a method for treating cancer, the method comprising administering a population of activated T-cells that comprise a polypeptide to a subject in need thereof, wherein said polypeptide comprises an antigen binding variable region comprising a CDR3 region, and wherein said CDR3 region comprises an amino acid sequence selected from the group consisting of SEQ ID NO:3, SEQ ID NO:7, SEQ ID NO:23, SEQ ID NO: 27, SEQ ID NO:43, and SEQ ID NO:47. In one embodiment, the cancer comprises a cancer cell expressing a FOXM1 protein presented on HLA-A*24:02. In another embodiment, the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell. In certain embodiments, a) the CDR3 region comprises the amino acid sequence of SEQ ID NO:3, and the antigen binding variable region further comprises a CDR1 region comprising the amino acid sequence of SEQ ID NO:1; b) the CDR3 region comprises the amino acid sequence of SEQ ID NO:3, and the antigen binding variable region further comprises a CDR2 region comprising the amino acid sequence of SEQ ID NO: 2; c) the CDR3 region comprises the amino acid sequence of SEQ ID NO:7, and the antigen binding variable region further comprises a CDR1 region comprising the amino acid sequence of SEQ ID NO:5; d) the CDR3 region comprises the amino acid sequence of SEQ ID NO: 7, and the antigen binding variable region further comprises a CDR2 region comprising the amino acid sequence of SEQ ID NO:6; e) the CDR3 region comprises the amino acid sequence of SEQ ID NO:23, and the antigen binding variable region further comprises a CDR1 region comprising the amino acid sequence of SEQ ID NO:21; f) the CDR3 region comprises the amino acid sequence of SEQ ID NO:23, and the antigen binding variable region further comprises a CDR2 region comprising the amino acid sequence of SEQ ID NO:22; g) the CDR3 region comprises the amino acid sequence of SEQ ID NO:27, and the antigen binding variable region further comprises a CDR1 region comprising the amino acid sequence of SEQ ID NO: 25; h) the CDR3 region comprises the amino acid sequence of SEQ ID NO:27, and the antigen binding variable region further comprises a CDR2 region comprising the amino acid sequence of SEQ ID NO:26; i) the CDR3 region comprises the amino acid sequence of SEQ ID NO: 43, and the antigen binding variable region further comprises a CDR1 region comprising the amino acid sequence of SEQ ID NO:41; j) the CDR3 region comprises the amino acid sequence of SEQ ID NO:43, and the antigen binding variable region further comprises a CDR2 region comprising the amino acid sequence of SEQ ID NO:42; k) the CDR3 region comprises the amino acid sequence of SEQ ID NO:47, and the antigen binding variable region further comprises a CDR1 region comprising the amino acid sequence of SEQ ID NO: 45; l) the CDR3 region comprises the amino acid sequence of SEQ ID NO:47, and the antigen binding variable region further comprises a CDR2 region comprising the amino acid sequence of SEQ ID NO:46; m) the CDR3 region comprises the amino acid sequence of SEQ ID NO: 3, and the antigen binding variable region further comprise an amino acid sequence having at least about 85% sequence identity to the amino acid sequence of SEQ ID NO:4; n) the CDR3 region comprises the amino acid sequence of SEQ ID NO:7, and the antigen binding variable region further comprise an amino acid sequence having at least about 85% sequence identity to the amino acid sequence of SEQ ID NO:8; o) the CDR3 region comprises the amino acid sequence of SEQ ID NO:23, and the antigen binding variable region further comprise an amino acid sequence having at least about 85% sequence identity to the amino acid sequence of SEQ ID NO:24;

p) the CDR3 region comprises the amino acid sequence of SEQ ID NO:27, and the antigen binding variable region further comprise an amino acid sequence having at least about 85% sequence identity to the amino acid sequence of SEQ ID NO:28; q) the CDR3 region comprises the amino acid sequence of SEQ ID NO:43, and the antigen binding variable region further comprise an amino acid sequence having at least about 85% sequence identity to the amino acid sequence of SEQ ID NO:44; or r) the CDR3 region comprises the amino acid sequence of SEQ ID NO:47, and the antigen binding variable region further comprise an amino acid sequence having at least about 85% sequence identity to the amino acid sequence of SEQ ID NO: 48.

In another aspect, the present disclosure provides a recombinant T-cell receptor comprising a TCRβ chain variable region and a TCRα chain variable region, wherein: a) the TCRβ chain variable region comprises a CDR3 region comprising the amino acid sequence of SEQ ID NO:3, and further comprises an amino acid sequence having at least about 85%, about 90%, or about 95% sequence identity to the amino acid sequence of SEQ ID NO:4, and wherein the TCRα chain variable region comprises a CDR3 region comprising the amino acid sequence of SEQ ID NO:7, and further comprises an amino acid sequence having at least about 85%, about 90%, or about 95% sequence identity to the amino acid sequence of SEQ ID NO:8; b) the TCRβ chain variable region comprises a CDR3 region comprising the amino acid sequence of SEQ ID NO:23, and further comprises an amino acid sequence having at least about 85%, about 90%, or about 95% sequence identity to the amino acid sequence of SEQ ID NO:24, and wherein the TCRα chain variable region comprises a CDR3 region comprising the amino acid sequence of SEQ ID NO:27, and further comprises an amino acid sequence having at least about 85%, about 90%, or about 95% sequence identity to the amino acid sequence of SEQ ID NO: 28; or c) the TCRβ chain variable region comprises a CDR3 region comprising the amino acid sequence of SEQ ID NO:43, and further comprises an amino acid sequence having at least about 85%, about 90%, or about 95% sequence identity to the amino acid sequence of SEQ ID NO: 44, and wherein the TCRα chain variable region comprises a CDR3 region comprising the amino acid sequence of SEQ ID NO:47, and further comprises an amino acid sequence having at least about 85%, about 90%, or about 95% sequence identity to the amino acid sequence of SEQ ID NO:48. In one embodiment, the TCRβ chain variable region comprises the amino acid sequence of SEQ ID NO:4, and wherein the TCRα chain variable region comprises the amino acid sequence SEQ ID NO:8. In another embodiment, the TCRβ chain variable region comprises the amino acid sequence of SEQ ID NO:24, and wherein the TCRα chain variable region comprises the amino acid sequence SEQ ID NO:28. In yet another embodiment, the TCRβ chain variable region comprises the amino acid sequence of SEQ ID NO:44, and wherein the TCRα chain variable region comprises the amino acid sequence SEQ ID NO:48. In still yet another embodiment, the recombinant T-cell receptor is a single chain T-cell receptor. In certain embodiments, the present disclosure provides a cell comprising a recombinant T-cell receptor of the present disclosure.

In yet another aspect, the present disclosure provides a composition comprising a T-cell receptor comprising a TCRβ chain variable region and a TCRα chain variable region, wherein: a) the TCRβ chain variable region comprises a CDR3 region comprising the amino acid sequence of SEQ ID NO:3, and further comprises an amino acid sequence having at least about 85%, about 90%, or about 95% sequence identity to the amino acid sequence of SEQ ID NO: 4, and wherein the TCRα chain variable region comprises a CDR3 region comprising the amino acid sequence of SEQ ID NO:7, and further comprises an amino acid sequence having at least about 85%, about 90%, or about 95% sequence identity to the amino acid sequence of SEQ ID NO:8; b) the TCRβ chain variable region comprises a CDR3 region comprising the amino acid sequence of SEQ ID NO:23, and further comprises an amino acid sequence having at least about 85%, about 90%, or about 95% sequence identity to the amino acid sequence of SEQ ID NO:24, and wherein the TCRα chain variable region comprises a CDR3 region comprising the amino acid sequence of SEQ ID NO:27, and further comprises an amino acid sequence having at least about 85%, about 90%, or about 95% sequence identity to the amino acid sequence of SEQ ID NO:28; or c) the TCRβ chain variable region comprises a CDR3 region comprising the amino acid sequence of SEQ ID NO:43, and further comprises an amino acid sequence having at least about 85%, about 90%, or about 95% sequence identity to the amino acid sequence of SEQ ID NO:44, and wherein the TCRα chain variable region comprises a CDR3 region comprising the amino acid sequence of SEQ ID NO:47, and further comprises an amino acid sequence having at least about 85%, about 90%, or about 95% sequence identity to the amino acid sequence of SEQ ID NO:48. In one embodiment, the TCRβ chain variable region comprises the amino acid sequence of SEQ ID NO:4, and wherein the TCRα chain variable region comprises the amino acid sequence SEQ ID NO:8. In another embodiment, the TCRβ chain variable region comprises the amino acid sequence of SEQ ID NO: 24, and wherein the TCRα chain variable region comprises the amino acid sequence SEQ ID NO: 28. In yet another embodiment, the TCRβ chain variable region comprises the amino acid sequence of SEQ ID NO:44, and wherein the TCRα chain variable region comprises the amino acid sequence SEQ ID NO:48. In still yet another embodiment, the T-cell receptor is a single chain T-cell receptor. In one embodiment, the composition comprises a plurality of cells comprising said T-cell receptor.

In some embodiments, the present disclosure provides a method of treating cancer, the method comprising administering a recombinant T-cell receptor, a cell, or a composition of the disclosure to a subject in need thereof. In one embodiment, the cancer comprises a cancer cell expressing a FOXM1 protein presented on HLA-A*24:02. Non-limiting examples of types of cancer cells that may be treated according to the methods of the present disclosure include a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

In still yet another aspect, the present disclosure provides a bi-specific T-cell engager comprising a TCRβ chain variable region and a TCRα chain variable region, wherein: a) the TCRβ chain variable region comprises a CDR3 region comprising the amino acid sequence of SEQ ID NO:3, and wherein the TCRα chain variable region comprises a CDR3 region comprising the amino acid sequence of SEQ ID NO:7; b) the TCRβ chain variable region comprises a CDR3 region comprising the amino acid sequence of SEQ ID NO:23, and wherein the TCRα chain variable region comprises a CDR3 region comprising the amino acid sequence of SEQ ID NO:27; or c) the TCRβ chain variable region comprises a CDR3 region comprising the amino acid sequence of SEQ ID NO:43, and wherein the TCRα chain variable region comprises a CDR3 region comprising the amino acid sequence of SEQ ID NO:47. In certain embodiments, a) the CDR3 region comprises the amino acid sequence of SEQ ID NO:3, and the antigen binding variable region further comprises a CDR1 region comprising the amino acid sequence of SEQ ID NO:1; b) the CDR3 region comprises the amino acid sequence of SEQ ID NO:3, and the antigen binding variable region further comprises a CDR2 region comprising the amino acid sequence of SEQ ID NO:2; c) the CDR3 region comprises the amino acid sequence of SEQ ID NO:7, and the antigen binding variable region further comprises a CDR1 region comprising the amino acid sequence of SEQ ID NO:5; d) the CDR3 region comprises the amino acid sequence of SEQ ID NO:7, and the antigen binding variable region further comprises a CDR2 region comprising the amino acid sequence of SEQ ID NO:6; e) the CDR3 region comprises the amino acid sequence of SEQ ID NO:23, and the antigen binding variable region further comprises a CDR1 region comprising the amino acid sequence of SEQ ID NO: 21; f) the CDR3 region comprises the amino acid sequence of SEQ ID NO:23, and the antigen binding variable region further comprises a CDR2 region comprising the amino acid sequence of SEQ ID NO:22; g) the CDR3 region comprises the amino acid sequence of SEQ ID NO: 27, and the antigen binding variable region further comprises a CDR1 region comprising the amino acid sequence of SEQ ID NO:25; h) the CDR3 region comprises the amino acid sequence of SEQ ID NO:27, and the antigen binding variable region further comprises a CDR2 region comprising the amino acid sequence of SEQ ID NO:26; i) the CDR3 region comprises the amino acid sequence of SEQ ID NO:43, and the antigen binding variable region further comprises a CDR1 region comprising the amino acid sequence of SEQ ID NO: 41; j) the CDR3 region comprises the amino acid sequence of SEQ ID NO:43, and the antigen binding variable region further comprises a CDR2 region comprising the amino acid sequence of SEQ ID NO:42; k) the CDR3 region comprises the amino acid sequence of SEQ ID NO: 47, and the antigen binding variable region further comprises a CDR1 region comprising the amino acid sequence of SEQ ID NO:45; l) the CDR3 region comprises the amino acid sequence of SEQ ID NO:47, and the antigen binding variable region further comprises a CDR2 region comprising the amino acid sequence of SEQ ID NO:46; m) the CDR3 region comprises the amino acid sequence of SEQ ID NO:3, and the antigen binding variable region further comprise an amino acid sequence having at least about 85% sequence identity to the amino acid sequence of SEQ ID NO:4; n) the CDR3 region comprises the amino acid sequence of SEQ ID NO:7, and the antigen binding variable region further comprise an amino acid sequence having at least about 85% sequence identity to the amino acid sequence of SEQ ID NO:8; o) the CDR3 region comprises the amino acid sequence of SEQ ID NO:23, and the antigen binding variable region further comprise an amino acid sequence having at least about 85% sequence identity to the amino acid sequence of SEQ ID NO:24; p) the CDR3 region comprises the amino acid sequence of SEQ ID NO:27, and the antigen binding variable region further comprise an amino acid sequence having at least about 85% sequence identity to the amino acid sequence of SEQ ID NO:28; q) the CDR3 region comprises the amino acid sequence of SEQ ID NO:43, and the antigen binding variable region further comprise an amino acid sequence having at least about 85% sequence identity to the amino acid sequence of SEQ ID NO: 44; or r) the CDR3 region comprises the amino acid sequence of SEQ ID NO:47, and the antigen binding variable region further comprise an amino acid sequence having at least about 85% sequence identity to the amino acid sequence of SEQ ID NO:48.

In certain embodiments, the present disclosure also provides cells and compositions comprising a bi-specific T-cell engager of the present disclosure. In one embodiment, the present disclosure provides a method of treating cancer, the method comprising administering the bi-specific T-cell engager of the present disclosure to a subject in need thereof. In another embodiment, the cancer comprises a cancer cell expressing a FOXM1 protein presented on HLA-A*24:02. In yet another embodiment, the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows results of testing PBMCs transduced with a FOXM1-specific TCR (TCR2, TCR3, TCR4) for specific lysis of wild-type 293 cells (white bars), 293 cells endogenously expressing FOXM1 and HLA-A*24:02 (black bars), or 293 cells endogenously expressing HLA-A*24:02 and pulsed with FOXM1 peptide (SEQ ID NO:64) (dark grey bars) at different effector cell to target cell ratios (40:1, 20:1, 5:1, and 2.5:1).

FIG. 2 shows the polypeptide (FIG. 2A) and nucleotide (FIG. 2B) sequences of the validated FOXM1/HLA-A*24:02-specific T-cell receptor TCR2. The sequences of the signal peptide are italicized, the sequences of the variable regions are in bold, the sequences of the CDR1, CDR2, and CDR3 regions are underlined, and the sequences of the linker peptide are in bold and italicized.

FIG. 3 shows the polypeptide (FIG. 3A) and nucleotide (FIG. 3B) sequences of the validated FOXM1/HLA-A*24:02-specific T-cell receptor TCR3. The sequences of the signal peptide are italicized, the sequences of the variable regions are in bold, the sequences of the CDR1, CDR2, and CDR3 regions are underlined, and the sequences of the linker peptide are in bold and italicized.

FIG. 4 shows the polypeptide (FIG. 4A) and nucleotide (FIG. 4B) sequences of the validated FOXM1/HLA-A*24:02-specific T-cell receptor TCR4. The sequences of the signal peptide are italicized, the sequences of the variable regions are in bold, the sequences of the CDR1, CDR2, and CDR3 regions are underlined, and the sequences of the linker peptide are in bold and italicized.

### BRIEF DESCRIPTION OF THE SEQUENCES

SEQ ID NO:1—An amino acid sequence of the CDR1 of a TCRβ chain of TCR2 that specifically binds epitopes of FOXM1.

SEQ ID NO:2—An amino acid sequence of the CDR2 of a TCRβ chain of TCR2 that specifically binds epitopes of FOXM1.

SEQ ID NO:3—An amino acid sequence of the CDR3 of a TCRβ chain of TCR2 that specifically binds epitopes of FOXM1.

SEQ ID NO:4—An amino acid sequence of the variable region of a TCRβ chain of TCR2 that specifically binds epitopes of FOXM1.

SEQ ID NO:5—An amino acid sequence of the CDR1 of a TCRα chain of TCR2 that specifically binds epitopes of FOXM1.

SEQ ID NO:6—An amino acid sequence of the CDR2 of a TCRα chain of TCR2 that specifically binds epitopes of FOXM1.

SEQ ID NO:7—An amino acid sequence of the CDR3 of a TCRα chain of TCR2 that specifically binds epitopes of FOXM1.

SEQ ID NO:8—An amino acid sequence of the variable region of a TCRα chain of TCR2 that specifically binds epitopes of FOXM1.

SEQ ID NO:9—An amino acid sequence of a TCRβ chain of TCR2 that specifically binds epitopes of FOXM1, which includes the sequences of both the variable region and the constant region.

SEQ ID NO:10—An amino acid sequence of a TCRα chain of TCR2 that specifically binds epitopes of FOXM1, which includes the sequences of both the variable region and the constant region.

SEQ ID NO:11—A nucleotide sequence that encodes SEQ ID NO:1.

SEQ ID NO: 12—A nucleotide sequence that encodes SEQ ID NO:2.

SEQ ID NO:13—A nucleotide sequence that encodes SEQ ID NO:3.

SEQ ID NO: 14—A nucleotide sequence that encodes SEQ ID NO:4.

SEQ ID NO:15—A nucleotide sequence that encodes SEQ ID NO:5.

SEQ ID NO:16—A nucleotide sequence that encodes SEQ ID NO:6.

SEQ ID NO:17—A nucleotide sequence that encodes SEQ ID NO:7.

SEQ ID NO:18—A nucleotide sequence that encodes SEQ ID NO:8.

SEQ ID NO:19—A nucleotide sequence that encodes SEQ ID NO:9.

SEQ ID NO:20—A nucleotide sequence that encodes SEQ ID NO:10.

SEQ ID NO:21—An amino acid sequence of the CDR1 of a TCRβ chain of TCR3 that specifically binds epitopes of FOXM1.

SEQ ID NO:22—An amino acid sequence of the CDR2 of a TCRβ chain of TCR3 that specifically binds epitopes of FOXM1.

SEQ ID NO:23—An amino acid sequence of the CDR3 of a TCRβ chain of TCR3 that specifically binds epitopes of FOXM1.

SEQ ID NO:24—An amino acid sequence of the variable region of a TCRβ chain of TCR3 that specifically binds epitopes of FOXM1.

SEQ ID NO:25—An amino acid sequence of the CDR1 of a TCRα chain of TCR3 that specifically binds epitopes of FOXM1.

SEQ ID NO:26—An amino acid sequence of the CDR2 of a TCRα chain of TCR3 that specifically binds epitopes of FOXM1.

SEQ ID NO:27—An amino acid sequence of the CDR3 of a TCRα chain of TCR3 that specifically binds epitopes of FOXM1.

SEQ ID NO:28—An amino acid sequence of the variable region of a TCRα chain of TCR3 that specifically binds epitopes of FOXM1.

SEQ ID NO:29—An amino acid sequence of a TCRβ chain of TCR3 that specifically binds epitopes of FOXM1, which includes the sequences of both the variable region and the constant region.

SEQ ID NO:30—An amino acid sequence of a TCRα chain of TCR3 that specifically binds epitopes of FOXM1, which includes the sequences of both the variable region and the constant region.

SEQ ID NO:31—A nucleotide sequence that encodes SEQ ID NO:21.

SEQ ID NO:32—A nucleotide sequence that encodes SEQ ID NO:22.

SEQ ID NO:33—A nucleotide sequence that encodes SEQ ID NO:23.

SEQ ID NO:34—A nucleotide sequence that encodes SEQ ID NO:24.

SEQ ID NO:35—A nucleotide sequence that encodes SEQ ID NO:25.

SEQ ID NO:36—A nucleotide sequence that encodes SEQ ID NO:26.

SEQ ID NO:37—A nucleotide sequence that encodes SEQ ID NO:27.

SEQ ID NO:38—A nucleotide sequence that encodes SEQ ID NO:28.

SEQ ID NO:39—A nucleotide sequence that encodes SEQ ID NO:29.

SEQ ID NO:40—A nucleotide sequence that encodes SEQ ID NO:30.

SEQ ID NO:41—An amino acid sequence of the CDR1 of a TCRβ chain of TCR4 that specifically binds epitopes of FOXM1.

SEQ ID NO:42—An amino acid sequence of the CDR2 of a TCRβ chain of TCR4 that specifically binds epitopes of FOXM1.

SEQ ID NO:43—An amino acid sequence of the CDR3 of a TCRβ chain of TCR4 that specifically binds epitopes of FOXM1.

SEQ ID NO:44—An amino acid sequence of the variable region of a TCRβ chain of TCR4 that specifically binds epitopes of FOXM1.

SEQ ID NO:45—An amino acid sequence of the CDR1 of a TCRα chain of TCR4 that specifically binds epitopes of FOXM1.

SEQ ID NO:46—An amino acid sequence of the CDR2 of a TCRα chain of TCR4 that specifically binds epitopes of FOXM1.

SEQ ID NO:47—An amino acid sequence of the CDR3 of a TCRα chain of TCR4 that specifically binds epitopes of FOXM1.

SEQ ID NO:48—An amino acid sequence of the variable region of a TCRα chain of TCR4 that specifically binds epitopes of FOXM1.

SEQ ID NO:49—An amino acid sequence of a TCRβ chain of TCR4 that specifically binds epitopes of FOXM1, which includes the sequences of both the variable region and the constant region.

SEQ ID NO:50—An amino acid sequence of a TCRα chain of TCR4 that specifically binds epitopes of FOXM1, which includes the sequences of both the variable region and the constant region.

SEQ ID NO:51—A nucleotide sequence that encodes SEQ ID NO:41.

SEQ ID NO:52—A nucleotide sequence that encodes SEQ ID NO:42.

SEQ ID NO:53—A nucleotide sequence that encodes SEQ ID NO:43.

SEQ ID NO:54—A nucleotide sequence that encodes SEQ ID NO:44.

SEQ ID NO:55—A nucleotide sequence that encodes SEQ ID NO:45.

SEQ ID NO:56—A nucleotide sequence that encodes SEQ ID NO:46.

SEQ ID NO:57—A nucleotide sequence that encodes SEQ ID NO:47.

SEQ ID NO:58—A nucleotide sequence that encodes SEQ ID NO:48.

SEQ ID NO:59—A nucleotide sequence that encodes SEQ ID NO:49.

SEQ ID NO:60—A nucleotide sequence that encodes SEQ ID NO:50.

SEQ ID NO:61—An amino acid sequence of a linker sequence that may join the TCRβ chain of SEQ ID NO:9 and the TCRα chain of SEQ ID NO:10, the TCRβ chain of SEQ ID NO: 29 and the TCRα chain of SEQ ID NO:30, or the TCRβ chain of SEQ ID NO:49 and the TCRα chain of SEQ ID NO:50.

SEQ ID NO:62—A nucleotide sequence that encodes SEQ ID NO:61.

SEQ ID NO:63—A representative amino acid sequence encoded by the human FOXM1 gene.

SEQ ID NO:64—An amino acid sequence of a representative antigenic peptide that of FOXM1.

### DETAILED DESCRIPTION OF THE INVENTION

The present disclosure provides antigen binding variable regions which specifically bind epitopes of FOXM1. The present disclosure further provides engineered cells, encoding nucleotide sequences, expression constructs, and associated methods of treating cancer which comprise or encode the antigen binding variable regions provided by the present disclosure.

### A. Engineered T Cell Receptors

T-cell receptors comprise two different polypeptide chains, termed the T-cell receptor a (TCRα) and β (TCRβ) chains, linked by a disulfide bond. Each TCRα and TCRβ chain comprises a variable antigen binding region, and the structure of these chains is similar to that of a Fab fragment of an immunoglobulin molecule. The TCRα and TCRβ chains are responsible for the antigen recognition demonstrated by most T-cells, although a minority of T-cells instead comprise an alternative but structurally similar TCR, which comprises a pair of polypeptide chains designated TCRγ and TCRδ. TCRαβ and TCRγδ receptors differ from membrane-bound immunoglobulins that serve as B-cell receptors in the following ways: 1) TCRs have only one antigen binding site, whereas immunoglobulins have two; and 2) TCRs are never secreted, whereas immunoglobulins may be secreted as an antibody.

Both chains of a TCR comprise an amino-terminal variable (V) region, a constant (C) region, and a short hinge region containing a cysteine residue that forms the interchain disulfide bond. The TCR V region shares homology with the immunoglobulin V domain, and the TCR C region shares homology with the immunoglobulin C domain. Each TCR chain comprises a hydrophobic transmembrane domain that spans the lipid bilayer and ends in a short cytoplasmic tail. In some embodiments, a TCR of the present disclosure may be a single chain TCR. Methods for producing single chain TCRs are known in the art and any such method may be used according to the embodiments of the present disclosure. Non-limiting examples of single chain TCRs are described in Knies, et al., Oncotarget 7(16): 21199-21221 and U.S. Pat. No. 10,538,573.

The three-dimensional structure of the TCR has been determined. The TCR chains fold in a manner similar to a Fab fragment, although the final 3D structure appears a little shorter and wider. There are, however, some distinct differences between TCRs and Fab fragments. The most striking difference is in the Ca domain, which folds differently than any other immunoglobulin-like domain. The half of the domain that is juxtaposed with the Cβ domain forms a β sheet similar to that found in other immunoglobulin-like domains, but the other half of the domain is formed of loosely packed strands and a short segment of a helix. The intramolecular disulfide bond of the Ca domain joins a β strand to this segment of a helix, however, in most immunoglobulin-like domains the intramolecular disulfide bond joins two β strands.

There are also differences in domain interaction between TCRs and immunoglobulins. The interface between the V and C domains of both TCR chains is more extensive than that demonstrated by antibodies, which may decrease the flexibility of the hinge region between the TCR domains. Furthermore, the interaction between the Cα and Cβ domains is distinct in that it is assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement. This displacement is particularly prevalent in the Vα CDR2 loop, which is oriented at roughly a right angle compared to the equivalent loop of an antibody V domain. This displacement is a result of a shift in the β strand that anchors one end of the loop from one face of the domain to the other. A strand displacement also causes a change in the orientation of the Vβ CDR2 loop in two of the seven Vβ domains for which structures are known.

In some aspects, the present disclosure provides recombinant polynucleotide molecules that encode a TCR antigen binding variable region. The present disclosure also provides recombinant polypeptides and recombinant or engineered cells comprising a TCR antigen binding variable region. As used herein the term “antigen binding variable region” refers to the region of a TCR chain which binds a pMHC. The antigen binding region of a TCR may comprise, in some embodiments, one, two, three, or four hypervariable complementarity-determining regions (CDRs). These CDRs form a binding site that specifically bind to a pMHC. The antigen binding variable region may comprise, for example, a CDR1 region, a CDR2 region, a CDR3 region, and/or a CDR4 region. The CDR3 region is particularly important for TCR-peptide binding. The phrase “specifically (or selectively) binds” or “specifically (or selectively) immunoreactive with,” when referring to a protein or peptide, refers to a binding reaction that is determinative of the presence of the protein or complex, often in a heterogeneous population of proteins or complexes. Thus, under typical immunoassay conditions, a specified antigen binding variable region may bind to a particular protein or complex at least two times the background. In specific embodiments, a specified antigen binding variable region may bind a particular protein or complex at least 2, at least 5, at least 10, at least 15, at least 20, at least 25, at least 30, at least 35, at least 40, at least 50, at least 55, at least 60, at least 65, at least 70, at least 75, at least 80, at least 85, at least 90, at least 95, or at least 100 times background, including any range derivable therebetween. Specific binding to an antigen binding variable region under such conditions requires an antigen binding variable region that is selected by virtue of its specificity for a particular protein or complex. A variety of assay formats known in the art may be used to select antigen binding variable regions specifically immunoreactive with a particular protein or complex and any such assay may be used to select an antigen binding variable region of the present disclosure. The antigen binding variable region of the present disclosure may specifically bind, in particular embodiments, a pMHC comprising a peptide derived from FOXM1. In one embodiment, the antigen binding variable region of the present disclosure may specifically bind a pMHC comprising a peptide derived from FOXM1 presented on HLA-A*24:02. In some embodiments, an antigen variable binding region of the present disclosure may cross-react with a small number of highly similar pMHCs.

As used herein, the term “recombinant” refers to a polynucleotide molecule, protein, or cell that is not naturally present, or is not naturally present in the same form or structure, and was created by human intervention. In one embodiment, a recombinant polynucleotide may be a DNA molecule or may be an RNA molecule. A recombinant polynucleotide molecule or a recombinant polypeptide molecule or protein may comprise, in certain embodiments, a combination of two or more polynucleotide or polypeptide sequences that do not naturally occur together in the same manner, such as a polynucleotide molecule or protein that comprises at least two polynucleotide or protein sequences that are operably linked but heterologous with respect to each other. As used herein the term “heterologous” refers to a polynucleotide molecule or protein that is not naturally present, or is not naturally present in the same form or structure, and was created by human intervention. For example, a heterologous polynucleotide molecule or protein may not naturally occur in the cell being transformed or may be expressed in a manner or genomic context that differs from the natural expression pattern or genomic context found in the cell being transformed. The heterologous polynucleotide molecule or protein, in some embodiments, may be overexpressed in the cell being transformed. In certain embodiments, a recombinant polynucleotide molecule, protein, construct, or vector may comprise any combination of two or more polynucleotide or protein sequences in the same molecule which are heterologous to one another, such that the combination is man-made and not normally found in nature. As used herein, the phrase “not normally found in nature” means not found in nature without human intervention. A recombinant polynucleotide or protein molecule, may comprise, for example, polynucleotide or protein sequences that are separated from other polynucleotide or protein sequences that exist in proximity to each other in nature. A recombinant polynucleotide or protein molecule may also comprise, for example, polynucleotide or protein sequences that are adjacent to or contiguous with other polynucleotide or protein sequences that are not naturally in proximity with each other. Such a recombinant polynucleotide molecule, protein, or expression construct may also refer to a polynucleotide or protein molecule or sequence that has been genetically engineered or constructed outside of a cell. For example, a recombinant polynucleotide molecule may comprise any engineered or man-made plasmid, vector, or expression construct, and may include a linear or circular DNA molecule. Such plasmids, vectors, and expression constructs may comprise, for example, various maintenance elements including, but not limited to, a heterologous promoter sequence, a prokaryotic origin of replication, or a selectable marker.

The present disclosure further provides engineered TCRs which comprise an antigen binding variable region of the present disclosure. As used herein the term “engineered TCR” refers to a TCR which comprises a recombinant polynucleotide molecule or a recombinant protein of the present disclosure. In some embodiments, an engineered TCR may comprise a chimeric polypeptide comprising an antigen binding variable region of the present disclosure and a heterologous TCR constant region. The engineered TCRs of the present disclosure may, in certain embodiments, specifically bind to the proteins and complexes described herein. The engineered TCRs of the present disclosure may specifically bind, in particular embodiments, a pMHC comprising a peptide derived from FOXM1. In one embodiment, the engineered TCRs of the present disclosure may specifically bind a pMHC comprising a peptide derived from FOXM1 presented on HLA-A*24:02. In some embodiments, the engineered TCRs of the present disclosure may cross-react with a small number of highly similar pMHCs. In some embodiments, the polynucleotide or protein sequences of the engineered TCRs of the present disclosure may comprise heterologous sequences that are used for cloning, enhanced expression, detection, or for therapeutic control of the recombinant polynucleotide molecule or that are not present in endogenous TCRs. Non-limiting examples of such heterologous sequences include, multiple cloning sites, linkers, hinge sequences, modified hinge sequences, modified transmembrane sequences, a polynucleotide or protein molecule used for detection, or therapeutic controls that allow for selection or screening of cells comprising the TCR. In some aspects of the present disclosure, the engineered TCR may comprise non-TCR sequences. Accordingly, certain aspects of the present disclosure relate to engineered TCRs comprising sequences that are not naturally found in TCRs. In certain embodiments, the engineered TCR is chimeric, and thus comprises, for example, sequences which are found normally found or encoded by a TCR gene.

In certain aspects, the present disclosure provides a Bi-specific T-cell engager (BiTE) comprising a TCR of the disclosure or antigen-binding fragment thereof. In some embodiments, a BiTE or TCR of the present disclosure is capable of binding epitopes derived from FOXM1. In one embodiment, a BiTE or TCR of the present disclosure is capable of binding epitopes derived from FOXM1 presented on HLA-A*24:02. In another embodiment, a BiTE or TCR of the present disclosure is capable of binding an epitope of FOXM1 comprising the sequence of SEQ ID NO:64.

As used herein a “bi-specific antibody molecule” refers to a molecule with two antigen binding domains, which may bind the same antigen or may bind different antigens. A BiTE is a subclass of bispecific antibody molecules. “Non-IgG-like” antibodies include antibodies that lack an Fc portion such as bispecific T cell engagers (BiTE), DART, tetravalent antiparallel structures (TandAbs) m and VH-only bi-nanobodies. The non-IgG-like antibodies due to their lack of an Fc portion, in some embodiments, may be smaller or have shorter in vivo half-lives.

As used herein, the term “Bi-specific T-cell engager” or “BiTE” refers to a class of artificial bispecific monoclonal antibodies that have been investigated for use as anti-cancer drugs. BiTEs direct the host's immune system, or more specifically the host's T-cells, to attack cancer cells. BiTEs are fusion proteins that may comprise, for example, two single-chain antigen binding variable fragments (scFvs) of different antibodies or TCRs, or amino acid sequences from four different genes, on a single peptide chain of about 55 kilodaltons. In certain embodiments, one of the scFvs may bind to T-cells through the CD3 receptor, and the other scFv may bind to a tumor cell through a tumor specific molecule. Unlike monospecific antibodies, BiTEs form a link between T-cells and tumor cells through their specificity for an antigen on the T-cell and an antigen on the tumor cell. This link results in a cytotoxic T-cell response against tumor cells and leads to the production of proteins such as perforin and granzymes, independent of the presence of MHC I or co-stimulatory molecules. These produced proteins enter tumor cells and initiate apoptosis, mimicking the physiological processes observed during T-cell-mediated responses against tumor cells. BiTE is a registered trademark of Micromet AG (a fully owned subsidiary of Amgen Inc).

In some aspects, a BiTE may comprise an antigen binding variable region, an antibody, or an antigen binding fragment that targets an antigen or a targeted epitope of interest expressed on the surface of a cell, such as a cancer cell, and also comprise an antigen binding variable region, an antibody, or an antigen binding fragment that specifically binds a CD3 coreceptor of a T-cell. In some aspects a BiTE of the present disclosure is capable of activating antigen specific T-cells, which kill target cancer cells expressing a particular epitope of interest. In one embodiment, a BiTE of the present disclosure activates T-cells that specifically bind an antigenic peptide derived from FOXM1. In another embodiment, a BiTE of the present disclosure activates T-cells that specifically bind an antigenic peptide derived from FOXM1 presented on HLA-A*24:02. Non-limiting examples of such an antigenic peptides include a peptide having the sequence of SEQ ID NO:64.

In some aspects, a BiTE of the present disclosure may be specific for at least one surface antigen on a T-cell of interest. Non-limiting examples of such T-cell surface antigens include CD3, CD2, VLA-1, CD8, CD4, CCR6, CXCR5, CD25, CD31, CD45RO, CD197, CD127, CD38, CD27, CD196, CD277 and CXCR3. In particular embodiments, a BiTE may comprise (i) an antigen binding region specific for a T-cell surface antigen, such as CD3, and (ii) an antigen binding region specific for an antigenic peptide derived from FOXM1. In one embodiment, the T-cell surface antigen may be selected from the group consisting of CD3 delta, CD3 epsilon, and CD3 gamma. In another embodiment, at least one antigen binding region of the BiTE may be specific for an antigenic peptide derived from FOXM1 presented on HLA-A*24:02. In yet another embodiment, the BiTE may comprise at least one antigen binding variable region of a TCRα chain or a TCRβ chain as described herein. In still yet another embodiment, the BiTE may comprise an antigen binding variable region of a TCRα chain and a TCRβ chain as described herein. In some aspects, the antigenic epitope of the target cell of interest may comprise an amino acid sequence peptide derived from FOXM1. In certain aspects, the antigenic epitope of the target cell may comprise the amino acid sequence of SEQ ID NO:64.

In certain aspects, an immune cell engager of the present disclosure, such as T-cell engager, may be arranged in the format VL1-linker1-VH1-linker1-VH2-linker3-VL2. In some embodiments, a BiTE of the present disclosure may bind an epitope comprising an amino acid sequence peptide derived from FOXM1 presented on HLA-A*24:02.

As used herein, the term “chimeric antigen receptor” or “CAR” refers to a chimeric receptor which utilizes the specificity of an antigen binding variable domain to direct T-cell activity to a target antigen of interest. In some embodiments, a CAR may comprise an antibody-derived antigen binding variable region. In other embodiments, a CAR may comprise a TCRα chain-derived and/or a TCRβ chain-derived antigen binding variable region. See, for example, Oh, et al., Sci Rep. 9:17291, 2019. In certain embodiments, a CAR may comprise an antigen binding variable region fused to a CD32 transmembrane and intracellular signaling domain, an antigen binding variable region fused to a CD28 transmembrane region with CD28 and CD32 intracellular signaling domains, or an antigen binding variable region fused to a CD28 transmembrane region with CD28, 4-1BB, and CD3ζ intracellular signaling domains. In many embodiments, a CAR may be a first generation, second generation, third generation, or any generation CAR.

In some aspects, a cell specifically recognized by an antigen binding variable region, a TCR, a BiTE, or a CAR of the present disclosure may be any cell which expresses or overexpresses FOXM1. In one embodiment, the cell is a cancer cell. In another embodiment, the cancer cell may be selected from the group consisting of: a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, a skin cancer cell, or any combination thereof. In particular embodiments, the cancer cell is selected from the group consisting of a lung cancer cell, a non-small cell lung cancer (NSCLC) cell, a breast cancer cell, or any combination thereof.

In some aspects, the engineered TCRs, BiTEs, or CARs provided by the present disclosure may comprise an amino acid sequence or be encoded by a nucleotide sequence comprising a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs: 1-62, or fragments thereof, including any range derivable therebetween. In certain embodiments, the engineered TCRs or BiTEs of the present disclosure comprise an antigen binding fragment which comprises or is encoded by a fragment of a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs: 1-62, or fragments thereof, including any range derivable therebetween. The antigen binding fragment may comprise, for example, at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275,300, 325, 350, 375, 400, 425, 450, 475, 500, 525, 550, 575, 600, 625, 650, 675, 700, 725, 750, 775, 800, 825, 850, 875, 900, 925, 950, 1000, 1100, 1200, 1300, 1400, 1500, 1600, 1700, or 1800 nucleotides or amino acid residues of a sequence having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs: 1-62, including any range derivable therebetween.

The terms “% identity” or “percent identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site found at ncbi.nlm.nih.gov/BLAST/or the like). Such sequences are then referred to as “substantially identical.” This definition also refers to, or applies to, the compliment of a particular sequence. The definition may also include sequences that have deletions, additions, and/or substitutions. Likewise, this definition also is intended to apply to complementarity between sequences.

In some aspects, a polynucleotide or polypeptide molecule provided by the present disclosure may comprise, may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 960, 97, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, or 1850 contiguous amino acid or nucleic acid residues of any one of SEQ ID NOS: 1-62, including all ranges derivable therebetween.

The polypeptides of the present disclosure may comprise, in certain embodiments, one or more amino acid substitutions. Any amino acid provided or encoded by any one of SEQ ID NOs: 1-62 may be substituted for any other amino acid.

In particular aspects, the engineered TCRs of the present disclosure comprise an amino acid sequence or are encoded by a nucleotide sequence comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 sequences having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs: 1-62, or fragments thereof, including all ranges derivable therebetween. In certain embodiments, the engineered TCRs of the present disclosure comprise an antigen binding fragment which comprises or is encoded by a sequence comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 sequences having at least about 70%, about 75%, about 80%, about 85%, about 90%, about 91%, about 92%, about 93%, about 94%, about 95%, about 96%, about 97%, about 98%, about 99%, or 100% sequence identity to any one of SEQ ID NOs: 1-62, or fragments thereof, including all ranges derivable therebetween.

In some embodiments, the recombinant polynucleotide or protein molecules of the present disclosure are based on a sequence found in humans. In one embodiment, the recombinant polynucleotide or protein molecules of the present disclosure comprise sequences or a combination of sequences which are not found in nature.

### B. Proteins and Protein Compositions

The present disclosure provides a number of polypeptide sequences that may be present, for example, in an antigen binding variable region as described herein. As used herein, the term “wild-type” refers to the endogenous version of a molecule that naturally occurs in an organism. In some aspects of the present disclosure, a wild-type version of a protein or polypeptide may be employed. In many aspects of the present disclosure, however, a recombinant protein or polypeptide is employed to generate an immune response. The terms recombinant protein and recombinant polypeptide may be used interchangeably with the terms modified protein, modified polypeptide, and variant. In certain embodiments, a recombinant protein refers to a protein having an altered chemical structure or amino acid sequence compared to the wild-type protein. In some aspects, a recombinant protein may have at least one modified activity or function compared to the wild-type protein. As is known in the art, proteins may have multiple activities or functions. In some embodiments, the function of a recombinant protein may be altered with respect to one activity or function, but retain the activity or function of the wild-type protein in other respects, such as immunogenicity. In certain embodiments, the proteins of the present disclosure may include those which comprise a mutation compared to the wild-type protein. In one embodiment, the mutation may comprise an insertion, a deletion, a truncation, or at least one amino acid substitution.

Where a protein is specifically mentioned herein, it may refer to a wild-type protein to a recombinant protein. In some embodiments, a protein of the present disclosure may have had its signal sequence removed. A protein of the present disclosure may be isolated from the organism in which it naturally occurs, produced by recombinant expression methods, or produced by solid-phase peptide synthesis (SPPS) or other in vitro methods known in the art.

The nucleotide and protein sequences for various genes have been previously disclosed, and may be found in computerized databases known in the art. Two such databases are the National Center for Biotechnology Information's GenBank and GenPept databases (on the World Wide Web at ncbi.nlm.nih.gov/) and The Universal Protein Resource (UniProt; on the World Wide Web at uniprot.org). The coding regions for these genes may be amplified or expressed using techniques known in the art or those disclosed herein.

The present disclosure provides compositions comprising the polypeptides of the present disclosure. In some embodiments, the compositions may comprise, for example, about 0.001 mg/ml to about 10.0 mg/ml of total polypeptide. The concentration of polypeptide may be, for example, 0.01 mg/ml to about 10.0 mg/ml, about 0.05 mg/ml to about 9.5 mg/ml, about 0.10 mg/ml to about 9.0 mg/ml, about 0.20 mg/ml to about 8.5 mg/ml, about 0.3 mg/ml to about 8.0 mg/ml, or at least 0.001 mg/ml, 0.010 mg/ml, 0.050 mg/ml, 0.10 mg/ml, 0.20 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.5 mg/ml, 2.0 mg/ml, 2.5 mg/ml, 3.0 mg/ml, 3.5 mg/ml, 4.0 mg/ml, 4.5 mg/ml, 5.0 mg/ml, 5.5 mg/ml, 6.0 mg/ml, 6.5 mg/ml, 7.0 mg/ml, 7.5 mg/ml, 8.0 mg/ml, 8.5 mg/ml, 9.0 mg/ml, 9.5 mg/ml, 10.0 mg/ml, including all ranges derivable therebetween. In certain embodiments, the composition may be serum-free, endotoxin-free, mycoplasma-free, and/or sterile.

As is known in the art, amino acid residues may be changed in a polypeptide sequence to create an equivalent, or even improved, second-generation variant polypeptide. For example, certain amino acids may be substituted for other amino acids in a polypeptide sequence without appreciable loss of binding capacity or specificity to structures such as, for example, binding sites on substrate molecules. Since the binding capacity, the binding specificity, and the nature of a protein define the functional activity of a protein, certain amino acid substitutions can be made in a protein sequence, and/or in its corresponding DNA coding sequence, such that the resultant variant protein comprises similar or desirable properties of the original protein. Thus, in some embodiments, the polynucleotide and polypeptide sequences of the present disclosure may comprise various amino acid or nucleic acid substitutions, deletions, and/or insertions without appreciable loss of biological utility or activity. As used herein the term “functionally equivalent codon” refers to codons that encode the same amino acid, such as the six different codons known in the art which code for arginine. As used herein, the terms “neutral substitutions” and “neutral mutations” refer to a change in a polypeptide sequence, or the encoding nucleotide sequence, such that the sequence comprises or encodes a biologically equivalent amino acid compared to that found in the original sequence. In certain embodiments, any amino acid of any polypeptide described herein may be substituted with any biologically equivalent amino acid. Biologically equivalent amino acids are known in the art.

Nucleic acid or amino acid sequence variants of the disclosure may comprise, in some embodiments, a substitution, an insertion, or a deletion. A polypeptide variant of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the polypeptide, as compared to the referenced polypeptide or to the wild-type polypeptide, including any range derivable therebetween. A variant polypeptide may comprise, for example, an amino acid sequence having at least 50%, 60%, 70%, 80%, or 90% sequence identity to a sequence comprising or encoded by any one of SEQ ID NOs: 1-62 or fragments thereof, including all ranges derivable therebetween. A polypeptide may include, for example, at least 1, 2, 3, 4, 5, 6, 7, 8, 9, 10, 11, 12, 13, 14, 15, 16, 17, 18, 19, or 20 amino acid substitutions.

It also will be understood that amino acid and nucleic acid sequences may include additional residues, such as additional N- or C-terminal amino acids, or 5′ or 3′ sequences, respectively, and yet still be essentially identical to the sequences provided by the present disclosure. Such essentially identical sequences, in some embodiments, may maintain the biological activity described herein for the sequences of the present disclosure. The addition of terminal sequences particularly applies to nucleic acid sequences that may, for example, include various non-coding sequences flanking either of the 5′ or 3′ portions of the coding region.

Deletion variants typically lack one or more amino acid residues compared to the protein from which the variant was derived, the native protein, or the wild-type protein. In certain embodiments, individual amino acid residues may be deleted, or a number of contiguous amino acids may be deleted. In one embodiment, a stop codon may be introduced, for example by substitution or insertion, into an encoding nucleic acid sequence to generate a truncated protein variant.

Insertional variants typically involve the addition of one or more amino acid residues at a non-terminal point of a polypeptide. Terminal additions may also be generated and can include fusion proteins. Non-limiting examples of such fusion proteins include multimers or concatemers of one or more polypeptides provided by the present disclosure.

Substitutional variants typically comprise the exchange of one amino acid for another at one or more sites within the polypeptide. Substitutional variants may be designed to modulate one or more properties of the polypeptide, with or without the loss of other functions or properties of the polypeptide. Amino acid substitutions may be conservative amino acid substitutions. As used herein, the term “conservative amino acid substitution” refers to an amino acid substitution wherein one amino acid is replaced with another amino acid having similar chemical properties. Conservative amino acid substitutions may involve, for example, the exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may, in some embodiments, encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be non-conservative. As used herein, the term “non-conservative amino acid substitution” refers to an amino acid substitution that affects a function of the polypeptide. Non-conservative amino acid substitutions typically involve substituting an amino acid residue with one that is chemically dissimilar. Non-conservative amino acid substitutions may include, for example, the substitution of a polar or charged amino acid for a nonpolar or uncharged amino acid, and vice versa. Non-conservative substitutions may also include, for example, the substitution of a member of one of the amino acid classes for a member from another class.

One skilled in the art can determine suitable polypeptide variants, as set forth herein, using well-known techniques. For example, one skilled in the art may identify suitable areas of the polypeptide molecule that may be changed without affecting activity by targeting regions not believed to be critical for activity. The skilled artisan will also be able to identify amino acid residues and portions of the polypeptide molecules that are conserved among similar proteins or polypeptides. In some embodiments, regions of a polypeptide molecule that may be important for biological activity or for structure may be subject to conservative amino acid substitutions without significantly altering the biological activity or adversely affecting the protein structure.

When making conservative or non-conservative amino acid substitutions, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the disclosure, those that are within ±1 are included, and in other aspects of the disclosure, those within ±0.5 are included.

As is known in the art, the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with the immunogenicity and antigen binding properties of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In some embodiments, amino acid substitutions based upon similar hydrophilicity values may include the substitution of amino acids whose hydrophilicity values are within ±2 of each other. In one embodiment, the substitution of amino acids whose hydrophilicity values are within ±1 or within ±0.5 are included. In some embodiments, a person of skill in the art may also identify epitopes from primary amino acid sequences based on hydrophilicity. These regions are also referred to as “epitopic core regions.” As is known in the art, an amino acid can be substituted for another having a similar hydrophilicity value and still produce a biologically equivalent and immunologically equivalent protein.

Additionally, it is well within the knowledge of a person of ordinary skill in the art to review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such amino acid residues predicted to be important.

One skilled in the art can also analyze the three-dimensional structure of a protein along with the amino acid sequence in relation to that structure in similar proteins. In view of such information, one skilled in the art may predict the alignment of amino acid residues of an antigen binding variable region or TCR with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments. This information may allow one skilled in the art to determine the amino acid positions where further substitutions should be avoided either alone or in combination with other substitutions. Various tools available to determine secondary structure can be found on the world wide web, for example, at expasy.org/proteomic s/protein structure.

In some aspects of the present disclosure, amino acid substitutions may be made that: (1) reduce susceptibility to proteolysis; (2) reduce susceptibility to oxidation; (3) alter binding affinity for forming protein complexes; (4) alter ligand or antigen binding affinities; and/or (5) confer or modify other physicochemical or functional properties of the polypeptides provided herein. For example, single or multiple amino acid substitutions may be made in the naturally occurring sequence. In one embodiment, the single or multiple amino acid substitutions may be conservative amino acid substitutions. Substitutions, in some embodiments, may be made in the portion of antigen binding variable region or the TCR that lie outside the domain(s) that form intermolecular contacts. In certain embodiments, conservative amino acid substitutions can be used that do not substantially change the structural characteristics of the protein. For example, one or more amino acid substitutions may be made that do not disrupt the secondary structure that characterizes the antigen binding variable region or TCR.

### C. Nucleic Acids

In certain aspects, the present disclosure provides polynucleotide molecules that encode the polypeptide molecules describes herein. Non-limiting example of such polynucleotide molecules include isolated polynucleotide segments, recombinant vectors, and recombinant polynucleotide molecules. The polynucleotide molecules may comprise, for example, sequences which encode one or more chains of an antigen binding variable region or a fragment thereof, sequences which encode a derivative, mutant, or variant of an antigen binding variable region or a fragment of thereof, or hybridization probes, PCR primers, or sequencing primers for identifying, analyzing, mutating, or amplifying a polynucleotide encoding a polypeptide. Polynucleotide molecule of the present disclosure may also include, for example, anti-sense polynucleotide molecules for inhibiting expression of a polynucleotide. Polynucleotide molecules that encode certain epitopes to which the antigen binding variable regions and TCRS described herein bind are also provided. Polynucleotide molecules encoding fusion proteins that include the polypeptides described herein are also provided. Polynucleotide molecules may be, in some embodiments, single-stranded or double-stranded, and may comprise RNA and/or DNA, or artificial variants thereof, for example peptide nucleic acids.

A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

By convention, the DNA sequences of the present disclosure and fragments thereof are disclosed with reference to only one strand of the two complementary DNA sequence strands. By implication and intent, the complementary sequences of the sequences provided here (the sequences of the complementary strand), also referred to in the art as the reverse complementary sequences, are within the scope of the disclosure and are expressly intended to be within the scope of the subject matter claimed. Thus, as used herein reference to any one of SEQ ID NOs: 11-20, 31-40, 51-60, or 62 and fragments thereof include and refer to the sequence of the complementary strand and fragments thereof.

A polynucleotide molecule of the present disclosure may, in some embodiments, comprise a contiguous nucleic acid sequence that encodes all or part of a polypeptide described herein. In certain embodiments, a polypeptide described herein may be encoded by variant nucleic acid sequences that encode the same or a substantially similar protein.

The polynucleotide molecules and fragments thereof provided by the present disclosure may, in some embodiments be combined with other polynucleotide molecules which comprise elements, such as promoters, polyadenylation signals, additional restriction enzyme sites, multiple cloning sites, other coding segments, and the like, such that the overall length of the polynucleotide molecule may vary considerably. The polynucleotide molecule provided by the present disclosure can be any length. In some cases, a nucleic acid sequence may encode a polypeptide sequence that comprises additional heterologous coding sequences. Such additional heterologous coding sequences may, for example, allow for purification, transport, secretion, post-translational modification, or for therapeutic benefits such as targeting or efficacy.

### D. Mutation

Changes introduced by mutation into a polynucleotide molecule may result in changes in the amino acid sequence of an encoded polypeptide, such as an antigen binding variable region or a TCR. Mutations can be introduced using any technique known in the art. In one embodiment, one or more selected amino acid residues may be changed using, for example, a site-directed mutagenesis protocol. In another embodiment, one or more random amino acid residues may be changed using, for example, a random mutagenesis protocol. Any resulting mutant polypeptide can be expressed and screened for desired properties, regardless of how it was created.

In certain embodiments, mutations may be introduced into a polynucleotide sequence without significantly altering the biological activity of its encoded polypeptide. For example, nucleotide substitutions may be made which result in amino acid substitutions at non-essential amino acid residues. In certain embodiments, one or more mutations may be introduced into a polynucleotide sequence that selectively change the biological activity of the encoded polypeptide (Romain Studer, et al., Biochem. J. 449:581-594 (2013)). In one embodiment, the mutation may quantitatively or qualitatively alter the biological activity of the encoded polypeptide. Non-limiting examples of such quantitative changes include increasing, reducing, or eliminating the activity of the encoded polypeptide. Non-limiting examples of such qualitative changes include altering the antigen specificity of an antigen binding variable region.

### E. Probes

In another aspect, the present disclosure provides polynucleotide molecules that are suitable for use as primers or hybridization probes for the detection of the polynucleotide molecules described herein. A polynucleotide molecule serving as a primer or probe, may comprise, in certain embodiments, only a portion or fragment of a nucleic acid sequence encoding a full-length polypeptide. Such primers or probes may be used, in certain embodiments, as probes or PCR primers for specific antigen binding variable region or TCR sequences. For instance, a nucleic acid molecule probe may be used in diagnostic methods. In certain embodiments, a nucleic acid molecule PCR primer may be used to amplify regions of DNA that could be used to isolate nucleic acid sequences for use in producing antigen binding variable regions. See, e.g., Gaily Kivi, et al., BMC Biotechnol. 16:2 (2016). In one embodiment, the nucleic acid molecules are oligonucleotides. In another embodiment, the oligonucleotides are from highly variable regions of a and β chains of the antigen binding variable region or TCR of interest. In still yet another embodiment, the oligonucleotides encode all or part of one or more of the CDRs or TCRs.

Probes based on the desired sequence of a nucleic acid molecule can be used to detect that nucleic acid molecule or similar nucleic acid molecules, for example, transcripts encoding a polypeptide of interest. Probes may comprise, in certain embodiments, a label, such as a radioisotope, a fluorescent compound, an enzyme, or an enzyme co-factor. In particular embodiments, the probes described herein may be used to identify a cell that expresses a polypeptide of interest.

### F. Antigenic Peptides

In some aspects, the present disclosure provides an antigenic peptide derived from a polypeptide encoded by the human FOXM1 gene. A representative polypeptide sequence encoded by the human FOXM1 gene is provided by SEQ ID NO:63. One of skill the art would understand that due to, for example, certain genetic polymorphisms or mutations that may be present in the human population, the encoded polypeptide may comprise a sequence having at least about 70%, about 75%, about 80%, about 85%, about 90%, about 91%, about 92%, about 93%, about 94%, about 95%, about 96%, about 97%, about 98%, about 99%, or 100% sequence identity to SEQ ID NO:63 or a fragment thereof. The antigenic peptide may be of any length that is capable of being presented by class I or class II MHC molecule. In particular embodiments, the antigenic peptide may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid residues in length. An antigenic peptide of the present disclosure may be isolated from the organism in which it naturally occurs, produced by recombinant expression methods, or produced by solid-phase peptide synthesis (SPPS) or other in vitro methods known in the art.

In certain embodiments, the present disclosure further relates to a method of identifying and isolating a TCR or antigen binding variable region according to the present disclosure. In one embodiment, the method may comprise the steps of incubating PBMCs from a healthy donor with an antigenic peptide as described herein, incubating the PBMCs with tetramer-phycoerythrin (PE) and isolating the high avidity T-cells by fluorescence activated cell sorting (FACS). In certain embodiments, the PBMCs from the healthy donor may present the antigenic peptide on HLA-A*24:02. In another embodiment, the present disclosure provides a method comprising obtaining a transgenic mouse with the entire human TCRαβ gene loci (1.1 and 0.7 Mb), whose T-cells express a diverse human TCR repertoire that compensates for mouse TCR deficiency, immunizing the mouse with an antigenic peptide described herein, incubating PBMCs obtained from the transgenic mice with tetramer-phycoerythrin (PE), and isolating the high avidity T-cells by fluorescence activated cell sorting (FACS).

### G. Polypeptide Expression

In some aspects, the present disclosure provides nucleic acid molecules encoding polypeptides described herein. In some embodiments, these nucleic acid molecules may encode a TCR antigen binding variable region, or an antigenic peptide as described herein. These nucleic acid molecules may be generated by any method known in the art. In some embodiments, the nucleic acids may be isolated from the organism in which they naturally occur, or produced using phage display. In certain embodiments, nucleic acid molecules may be expressed in any suitable recombinant expression system and allowed to assemble to form molecules which comprise an antigen binding variable region, or an antigenic peptide as described herein. The nucleic acid molecules may be used to express large quantities of polypeptides. If the nucleic acid molecules are derived from a non-human animal, the nucleic acid molecules may be used for humanization of the TCR genes.

### I. Vectors

In some aspects, the present disclosure provides expression vectors comprising a nucleic acid molecule encoding a polypeptide of a desired sequence or a portion thereof. In particular embodiments, the nucleic acid molecule may encode a fragment comprising one or more CDRs or one or more variable region domains. Expression vectors comprising the nucleic acid molecules may encode an a chain, a β chain, or an antigen-binding variable region thereof. In some aspects, expression vectors comprising nucleic acid molecules may encode fusion proteins, modified TCRs, or TCR fragments. In certain embodiments, the expression vectors described herein may comprise control sequences that govern transcription and translation, as well as nucleic acid sequences that serve other functions. In particular embodiments, polypeptides or peptides of the disclosure may be expressed when polynucleotide molecules of the present disclosure are inserted into expression vectors such that the coding sequence is operably linked to transcriptional and translational control sequences. As used herein, the term “operably linked” refers to at least two nucleotide molecules arranged or linked in a manner so that one can affect the function of the other. The two nucleotide molecules can be part of a single contiguous nucleotide molecule and can be adjacent or separated. In some aspects, the present disclosure provides a vector that encodes a functionally complete TCR sequence with appropriate restriction sites engineered so that any variable region sequences can be inserted and expressed. In particular embodiments, the present disclosure provides a vector that encodes a functionally complete human TCRα or TCRβ sequence with appropriate restriction sites engineered so that any variable sequence, or any CDR1, CDR2, and/or CDR3 encoding sequence can be inserted and expressed. Expression vectors may be used in any host cell and may, in some embodiments, contain sequences for plasmid or virus maintenance, or for cloning and expression of exogenous nucleotide sequences. In certain embodiments, such sequences, collectively referred to as “flanking sequences” may include one or more of the following operably linked nucleotide sequences: a promoter, an enhancer sequence, an origin of replication, a transcriptional termination sequence, a complete intron sequence containing a donor and acceptor splice site, a sequence encoding a leader sequence for polypeptide secretion, a ribosome binding site, a “polyadenylation sequence, a polylinker region for inserting the nucleic acid encoding the polypeptide to be expressed, or a selectable marker element. Such sequences and methods of using the same are well known in the art.

### II. Expression Systems

Numerous expression systems exist that comprise at least a part or all of the expression vectors discussed above. Prokaryote- and/or eukaryote-based systems can be employed for use to produce nucleic acid sequences provided by the present disclosure, or their encoded polypeptides, proteins, and peptides. Commercially and widely available systems include but are not limited to bacterial, mammalian, yeast, and insect cell systems. Different host cells have characteristic and specific mechanisms for the post-translational processing and modification of proteins. Appropriate cell lines or host systems can be chosen to ensure correct modification and processing of the foreign protein expressed. Those skilled in the art are able to express a vector to produce a nucleic acid sequence or its encoded polypeptide, protein or peptide using an appropriate expression system.

### III. Methods of Gene Transfer

Suitable methods for nucleic acid delivery to a host cell, tissue, or organism are well-known in the art, and any such methods may be used to introduce a nucleic acid provided by the present disclosure. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe, et al., 1990); by using DEAE dextran followed by polyethylene glycol (Gopal, 1985); by direct sonic loading (Fechheimer, et al., 1987); by liposome mediated transfection (Nicolau and Sene, 1982; Fraley, et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler, et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh, et al., 1993; U.S. Pat. Nos. 4,684,611 and 4,952,500, each incorporated herein by reference); by desiccation/inhibition mediated DNA uptake (Potrykus, et al., 1985). Other methods include viral transduction, such as gene transfer by lentiviral or retroviral transduction.

### IV. Host Cells

In another aspect, the present disclosure provides host cells comprising an expression vector as described herein. Antigen binding variable domains, TCRs, and BiTEs can be expressed in a variety of cell types, and the nucleic acids and proteins of the present disclosure may be expressed in any cell type known in the art. In some embodiments, an expression construct encoding antigen binding variable region, TCR, or BiTE may be transfected into any host cell known in the art using any of the variety of methods known in the art and described herein. Expression vectors may be introduced, in some embodiments, into prokaryotic or eukaryotic cells using any transformation or transfection technique known in the art. Some expression vectors may comprise control sequences that allow for replication and/or expression in both prokaryotic and eukaryotic cells. In certain aspects, the expression vectors described herein may be under control of a promoter that is linked to T-cell activation. Such promoters include but are not limited to those controlled by the transcription factor NFAT-1 or NF-Kβ. Both NFAT-1 or NF-κB may be activated upon T-cell activation. Control of antigen binding variable region, TCR, or BiTE expression allows T cells, such as tumor-targeting T cells, to sense their surroundings and perform real-time modulation of cytokine signaling, both in the T cells themselves, and in surrounding endogenous immune cells. Conditions under which to incubate host cells in order to maintain them and to permit replication of a vector are well-known in the art. Also understood and known in the art are techniques and conditions that allow for large-scale vector, nucleic acid, and polypeptide production, and any such techniques may be used to produce the vectors, polynucleotide molecules, and polypeptides described herein.

As is known in the art, mammalian cells may be stably transfected with a nucleic acid molecule of interest. During stable transfection, in certain embodiments, only a small fraction of cells may integrate the foreign DNA into their genome. In order to identify and select these stable transfectants, a selectable marker may be introduced into the host cells along with the gene of interest. In one embodiment, the selectable marker may be a gene that confers resistance to antibiotics. Stable transfectants may, in certain embodiments, be identified using drug selection or by any other method known in the art.

### H. Activated T-Cells

In certain aspects, the present disclosure provides activated T-cells which comprise a polynucleotide or polypeptide sequence described herein. In certain embodiments, an activated T-cell of the present disclosure may selectively bind or recognize a pMHC comprising an antigenic peptide. The recognized pMHC complex may, in particular embodiments, be a pMHC complex comprising an antigenic peptide derived from a polypeptide encoded by the human FOXM1 gene. In one embodiment, activated T-cells of the present disclosure may specifically bind an antigenic peptide derived from FOXM1 presented on HLA-A*24:02.

Activated T-cell may be produced by any in vitro, ex vivo, or in vivo method known in the art. In some embodiments, activated T-cell may be produced by contacting T-cells with peptide loaded human class I or II MHC molecules for a period of time sufficient to activate the T cells. In particular embodiments, activated T-cell may be produced by contacting T-cells with peptide loaded human class I or II MHC molecules expressed on the surface of a cell for a time period sufficient to activate the T-cells. In one embodiment, the cell on which the peptide-loaded human class I or II MHC molecules are expressed is an antigen-presenting cell. In another embodiment, an antigenic peptide provided by the present disclosure presented on HLA-A*24:02 may be used to produce activated T-cells. In certain embodiments, the time period sufficient to activate T-cells may be less than, greater than, or equal to about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 54 hours, about 60 hours, about 66 hours, about 72 hours, about 1 day, about 2 days, about 3 days, about 4 days, about 5 days, about 6 days, about 7 days, about 8 days, about 9 days, about 10 days, about 11 days, about 12 days, about 13 days, or about 14 days, including all ranges derivable therebetween.

In one aspect, activated T cells, which are produced by the above method, will specifically recognize a cell that expresses an antigenic peptide as described herein. In certain embodiments, the activated T-cell recognizes the cell by interacting through its TCR with an HLA/peptide-complex as described herein. T-cells are useful in a method of killing target cells in a patient whose target cells express a polypeptide comprising an antigenic peptide as described herein. The T-cells that are administered to the patient may be, in some embodiments, derived from the patient and activated as described above. In particular embodiments, the T-cells are not from the patient but are from another individual. In one embodiment, the individual is a healthy individual or donor. As used herein the terms, “healthy individual” or “healthy donor” refers to an individual that is generally in good health, preferably has a competent immune system and, more preferably, is not suffering from any disease that can be readily tested for and detected.

Complexes of peptide and MHC class I are recognized by CD8-positive T cells bearing the appropriate TCR, whereas complexes of peptide and MHC class II molecules are recognized by CD4-positive-helper-T cells bearing the appropriate TCR. It is well known that the TCR, the peptide and the MHC are thereby present in a stoichiometric amount of 1:1:1.

CD4-positive helper T-cells play an important role in inducing and sustaining effective responses by CD8-positive cytotoxic T-cells. At the tumor site, T-helper cells, support a cytotoxic T cell-(CTL-) friendly cytokine environment and attract effector cells, such as CTLs, natural killer (NK) cells, macrophages, and granulocytes.

In the absence of inflammation, the expression of MHC class II molecules is mostly restricted to cells of the immune system, especially professional antigen-presenting cells (APC), such as monocytes, monocyte-derived cells, macrophages, dendritic cells. In cancer patients, cells of the tumor have been found to express MHC class II molecules (Dengjel, et al., 2006).

T-helper cells, activated by MHC class II epitopes, play an important role in orchestrating the effector function of CTLs in anti-tumor immunity. T-helper cell epitopes that trigger a T-helper cell response of the TH1 type support effector functions of CD8-positive killer T cells, which include cytotoxic functions directed against tumor cells displaying tumor-associated peptide/MHC complexes on their cell surfaces. In this way, tumor-associated T-helper cell peptide epitopes, alone or in combination with other tumor-associated peptides, can serve as active pharmaceutical ingredients of compositions that stimulate anti-tumor immune responses.

It was shown in mammalian animal models that even in the absence of CD8-positive T lymphocytes, CD4-positive T cells are sufficient for inhibiting manifestation of tumors through inhibition of angiogenesis by secretion of interferon-gamma (IFNγ) (Beatty and Paterson, 2001; Mumberg, et al., 1999). There is also evidence for CD4 T cells as direct anti-tumor effectors (Braumuller, et al., 2013; Tran, et al., 2014).

Since the constitutive expression of HLA class II molecules is usually limited to immune cells, the possibility of isolating class II peptides directly from primary tumors was previously not considered possible. However, Dengjel, et al. were successful in identifying a number of MHC Class II epitopes directly from tumors (WO 2007/028574).

Since both types of response, CD8 and CD4 dependent, contribute jointly and synergistically to the anti-tumor effect, the identification and characterization of tumor-associated antigens recognized by either CD8+ T cells (ligand: MHC class I molecule+peptide epitope) or by CD4-positive T-helper cells (ligand: MHC class II molecule+peptide epitope) is important in the development of tumor immunotherapy.

For an MHC class I peptide to elicit a cellular immune response, it also must bind to an MHC-molecule. This process is dependent on the allele of the MHC-molecule and specific polymorphisms of the amino acid sequence of the peptide. MHC-class-1-binding peptides are usually 8-12 amino acid residues in length and usually contain two conserved residues (“anchors”) in their sequence that interact with the corresponding binding groove of the MHC-molecule. In this way each MHC allele has a “binding motif” determining which peptides can bind specifically to the binding groove.

In the MHC class I dependent immune reaction, peptides not only have to be able to bind to certain MHC class I molecules expressed by tumor cells, but they subsequently also have to be recognized by T cells bearing specific T cell receptors (TCR).

In vivo, the target cells for the T-cells according to the present disclosure can be cells of the tumor, which may express MHC class I or MHC Class II molecules and/or stromal cells surrounding the tumor cells, which may also express MHC class II molecules (Dengjel, et al., 2006). The T-cells of the present disclosure, in certain embodiment, may be used as active ingredients of a therapeutic composition.

In some embodiments, the present disclosure provides a method of treating a subject in need thereof comprising administering to the subject a population of activated T-cells or T-cells that comprise a polynucleotide molecule or a polypeptide molecule of the present disclosure. In one embodiment the activated T-cells are autologous to the patient or the subject in need thereof. In another embodiment, the activated T-cells are obtained from a healthy donor. In yet another embodiment, the activated T-cells are isolated from a human or animal subject using the methods described herein. In yet another embodiment, the activated T-cells have been transfected with a polynucleotide molecule as described herein.

### I. Pharmaceutical and Therapeutic Compositions

In certain aspects, the present disclosure provides pharmaceutical and therapeutic compositions comprising the polynucleotide molecules, antigen binding variable regions, TCRs, and/or cells of the present disclosure. In some embodiments, the polynucleotide molecules, antigen binding variable regions, TCRs, and/or cells of the present disclosure may be combined with a pharmaceutically acceptable carrier. As used herein, a “pharmaceutically acceptable carrier,” “pharmaceutically acceptable adjuvant,” or “adjuvant” refers to reagents, cells, compounds, materials, compositions, and/or dosage forms that are not only compatible with the polynucleotide molecules, polypeptide molecules, cells, and/or or other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Also included may be an agent that modifies the effect of other agents and is useful in preparing a therapeutic compound or composition that is generally safe, non-toxic, and neither biologically nor otherwise undesirable. Such an agent may be added to a therapeutic composition to modify the immune response of a subject by boosting the response such as to give a higher amount of polynucleotide molecules, polypeptide molecules, and or cells and longer-lasting protection from degradation. Such an agent may include any excipient, diluent, carrier, or adjuvant that is acceptable for pharmaceutical use. Such an agent may be non-naturally occurring, or may be naturally occurring, but not naturally found in combination with other agents in the immunogenic composition.

As used herein, a “therapeutic compound” or “therapeutic composition” refers to a composition comprising an antigen binding variable region, a TCR, or a cell of the present disclosure. In some embodiments, a therapeutic composition has the activity of specifically binding an antigenic peptide pMHC complex as described herein. In one embodiment, the composition is capable of eliciting an immune response. Such a compound or composition is meant to encompass a composition suitable for administration to a subject, such as a mammal, particularly a human subject. In general, a therapeutic composition is sterile, and preferably free of contaminants that are capable of eliciting an undesirable response within the subject (e.g., the compound(s) in the immunogenic composition is pharmaceutical grade). Therapeutic compositions may be designed for administration to subjects in need thereof via a number of different routes of administration including oral, intravenous, buccal, rectal, parenteral, intraperitoneal, intradermal, intratracheal, intramuscular, subcutaneous, inhalational, and the like. The appropriate dosage of a composition, as described herein, may be determined based on the type of disease to be treated, the severity and course of the disease, the clinical condition of the individual, clinical history, response to the treatment, and the discretion of the attending physician. In some embodiments, therapeutic compositions provided by the present disclosure may include various “unit doses.” A unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

As used herein, “subject” or “patient” refers to animals, including humans, who are treated with the therapeutic compounds or compositions or in accordance with the methods described herein. For diagnostic or research applications, a wide variety of mammals may be suitable subjects, including rodents (e.g., mice, rats, hamsters), rabbits, primates, and swine, such as inbred pigs and the like. In particular embodiments, a subject in need of therapy may be any subject who comprises a cell that expresses or overexpresses FOXM1. In one embodiment, the cell is a cancer cell. In another embodiment, the cancer cell may be selected from the group consisting of: a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, a skin cancer cell. In particular embodiments, the cancer cell is selected from the group consisting of a lung cancer cell, a non-small cell lung cancer (NSCLC) cell, a breast cancer cell.

A composition, as described herein, may include, in particular embodiments, a combination of therapeutic agents. In some embodiments, a composition as described here may be administered as a single composition or as more than one composition. Different compositions as provided herein, in certain embodiments, may be administered by the same route of administration or by different routes of administration.

In certain embodiments, the compositions and methods for treating an individual described herein may be combined with any other composition or method of treatment known in the art. The compositions and methods may be administered in any suitable manner known in the art. For example, a first and a second cancer treatment may be administered sequentially (at different times) or concurrently (at the same time). In some aspects, a first and a second cancer treatment may be administered in separate compositions. In certain embodiments, a first and a second cancer treatment may be administered in the same composition.

Non-limiting examples of additional treatment modalities that may be included in combination with the compositions and methods provided herein include immunotherapy, chemotherapy, radiation therapy, and surgery. In specific embodiments, the methods and compositions of the present disclosure may be combined with other therapies directed towards FOXM1, non-limiting examples of which include thiostrepton (TST), siomycin A, Robert Costa Memorial drug-1 (RCM-1), and Forkhead domain inhibitor-6 (FDI-6). Types of immunotherapy that may be used in combination with the compositions and methods provided by the present disclosure include, but are not limited, to: (a) checkpoint inhibitors, for example, inhibitors of PD-1, PDL1, PDL2, and CTLA-4; (b) inhibitors of co-stimulatory molecules, for example, inhibitors of B7-1, B7-2 CD28, ICOS, OX40, 4-1BB, CD137, CD40L, and GITR; (c) dendritic cell therapy; (d) other T-cell therapies directed to the same or to a different antigen; (e) cytokine therapy. Non-limiting types of chemotherapy that may be used in combination with the compositions and methods provided by the present disclosure include (a) alkylating agents; (b) antimetabolites; (c) natural products, for example, vinca alkaloids, enzymes (e.g., L-asparaginase), and biological response modifiers (e.g., interferon-α); and (d) other agents, for example, platinum coordination complexes, substituted ureas, and taxols.

### J. Detection and Therapeutic Agents

In some aspects, the present disclosure provided methods and compositions for detection and therapeutic labeling of the antigen binding variable domains, TCRs, fusion proteins, and/or cells provided by the present disclosure. Methods for labeling and detection of polypeptide molecules are well-known in the art, and any such method known in the art may be used to label or detect the polypeptides described herein. As a non-limiting example, polypeptides may be labeled with a detectable moiety, such as a radioactive atom, a chromophore, a fluorophore, or the like, and then detected using methods known in the art. Such labeled polypeptides may be used, in some embodiments, for in vivo or in vitro diagnostic techniques.

As used herein, the term “label” refers to a directly or indirectly detectable compound or composition that is conjugated directly or indirectly to the composition to be detected. In certain embodiments, a polynucleotide molecule, protein, or cell may be labeled to generate a labeled composition. In particular embodiments, labeled compositions also include sequences which are conjugated a polynucleotide molecule that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition that is detectable. Labels may be suitable for small scale detection or for high-throughput screening. As such, suitable labels include, but are not limited to radioisotopes, fluorochromes, chemiluminescent compounds, dyes, and proteins, including enzymes. Labels may be simply detected or may be quantified. In certain embodiments, labels that may be quantified provide numerically reportable value. In luminescence or fluorescence assays, the detectable response may be generated directly using a luminophore or fluorophore associated with an assay component involved in binding, or indirectly using a luminophore or fluorophore associated with another (e.g., reporter or indicator) component.

### K. Formulations and Culture of the Cells

In particular aspects, the cells of the disclosure may be cultured in a particular medium. The medium, in certain aspects, may be prepared using a medium used for culturing mammalian cells as their basal medium. Non-limiting examples of such a medium include such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, aMEM, DMEM, Ham, RPMI-1640, and Fischer's media, as well as any combinations thereof. In one embodiment, the medium may be xeno-free or chemically defined.

The medium, in particular embodiments, may be a serum-containing, serum-free, or xeno-free. In some embodiments, serum may be derived from the same animal as that of the cells being cultured to prevent contamination with heterogeneous animal-derived components. Serum-free medium refers to a medium with no unprocessed or unpurified serum and accordingly, can include medium with purified blood-derived components or animal tissue-derived components (such as growth factors).

In certain embodiments, the medium may contain or may not contain a serum alternative. Alternatives to serum may include, in one embodiment, one or more of the following: albumin (such as lipid-rich albumin, bovine albumin, recombinant albumin, humanized albumin, plant starch, dextran, or protein hydrolysates), transferrin (or other iron transporters), fatty acids, insulin, collagen precursors, trace elements, 2-mercaptoethanol, 3′-thioglycerol, or equivalents thereof. The alternatives to serum can be prepared by the method disclosed in International Publication No. WO 98/30679, for example, (incorporated herein in its entirety). Alternatively, any commercially available materials can be used for more convenience. Such commercially available materials include, but are not limited to, Knockout Serum Replacement (KSR), Chemically defined Lipid Concentrated (Gibco), and Glutamax (Gibco).

In certain aspects, the medium may comprise at least of the following: (1) vitamins such as biotin, DL alpha-tocopherol acetate, DL alpha-tocopherol, or vitamin A; (2) proteins such as BSA (bovine serum albumin), human albumin, fatty acid free fraction V, catalase, human recombinant insulin, human transferrin, or superoxide dismutase; or (2) other components such as corticosterone, D-galactose, ethanolamine HCl, glutathione (reduced), L-carnitine HCl, linoleic acid, progesterone, putrescine 2HCI, sodium selenite, or T3 (triodo-I-thyronine). In specific aspects, one or more of the components listed above may be explicitly excluded.

In some aspects, the medium comprises vitamins. In particular embodiments, the medium comprises at least one of the following: biotin, DL alpha-tocopherol acetate, DL alpha-tocopherol, vitamin A, choline chloride, calcium pantothenate, pantothenic acid, folic acid, nicotinamide, pyridoxine, riboflavin, thiamine, inositol, vitamin B12, or the medium may include combinations thereof or salts thereof. In some aspects, the medium comprises or consists essentially of biotin, DL alpha-tocopherol acetate, DL alpha-tocopherol, vitamin A, choline chloride, calcium pantothenate, pantothenic acid, folic acid, nicotinamide, pyridoxine, riboflavin, thiamine, inositol, and vitamin B12. In some aspects, the vitamins include or consist essentially of biotin, DL alpha-tocopherol acetate, DL alpha-tocopherol, vitamin A, or combinations or salts thereof. In some aspects, the medium further comprises proteins. In certain embodiments, the proteins comprise albumin or bovine serum albumin, a fraction of BSA, catalase, insulin, transferrin, superoxide dismutase, or combinations thereof. In some aspects, the medium further comprises one or more of the following: corticosterone, D-dalactose, ethanolamine, glutathione, L-carnitine, linolenic acid, progesterone, putrescine, sodium selenite, or triodo-I-thyronine, or combinations thereof. In some aspects, the medium comprises one or more of the following: a B-27® supplement, xeno-free B-27® supplement, GS21TM supplement, or combinations thereof. In one embodiment, the medium comprises or further comprises amino acids, monosaccharides, inorganic ions. In another embodiment, the amino acids comprise arginine, cystine, isoleucine, leucine, lysine, methionine, glutamine, phenylalanine, threonine, tryptophan, histidine, tyrosine, or valine, or combinations thereof. In yet another embodiment, the inorganic ions comprise sodium, potassium, calcium, magnesium, nitrogen, or phosphorus, or combinations or salts thereof. In still yet another embodiment, the medium further comprises one or more of the following: molybdenum, vanadium, iron, zinc, selenium, copper, or manganese, or combinations thereof.

In certain embodiments, one or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, including any range derivable therebetween.

In specific aspects, the cells of the disclosure are specifically formulated. In one embodiment, the cells may or may not be formulated as a cell suspension. In another embodiment, the cells may be formulated in a single dose form. In certain embodiments, the cells may be formulated for systemic or local administration. In particular embodiments, the cells are formulated for storage prior to use, and the cell formulation may comprise one or more cryopreservation agents, such as DMSO (for example, in 5% DMSO). In one embodiment, the cell formulation may comprise albumin, including human albumin, with a specific formulation comprising 2.5% human albumin. In another embodiment, the cells may be formulated specifically for intravenous administration. In specific embodiments, the cells are formulated for intravenous administration over less than one hour. In particular aspects the cells are in a formulated cell suspension that is stable at room temperature for at least about 1, 2, 3, or 4 hours from time of thawing.

In particular aspects, the cells of the disclosure comprise an exogenous TCR, which may be of a defined antigen specificity. In some aspects, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR. In some aspects, the choice of exogenous TCR may not necessarily be defined based on lack of alloreactivity. In certain embodiments, the endogenous TCR genes have been modified by genome editing so that they do not express a protein. Methods of gene editing such as methods using the CRISPR/Cas9 system are known in the art and described herein.

In some aspects, the cells of the disclosure comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, folate receptor-a, FAP, FBP, fetal AchR, FOXM1, FRα, GD2, G250/CAIX, GD3, Glypican-3 (GPC3), Her2, IL-13Rα2, Lambda, Lewis-Y, Kappa, KDR, MAGE, MCSP, Mesothelin, Muc 1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pme117, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two non-identical antigens, or it may be specific for more than two non-identical antigens.

### L. Kits

Certain aspects of the present disclosure further provide kits containing compositions of the disclosure or compositions to implement methods of the invention. In some aspects, kits can be used to evaluate one or more biomarkers or HLA types. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise, in certain embodiments, components, which may be individually packaged or placed in a container, such as a tube, bottle, vial, syringe, or other suitable container means.

Individual components, in particular embodiments, may also be provided in a kit in concentrated amounts. In one embodiment, a component is provided individually in the same concentration as it would be in a solution with other components. In certain embodiments, concentrations of components may be provided as 1×, 2×, 5×, 10×, or 20× or more.

In certain aspects, negative and/or positive control nucleic acids, probes, and inhibitors are included in some kit aspects. In some embodiments, a kit may include a sample that is a negative or positive control for methylation of one or more biomarkers.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components. As used herein, the term “consists essentially of,” when used in reference to a nucleotide or amino acid sequence of the present disclosure, means that the nucleotide sequence or amino acid sequence, may contain additional nucleotides or amino acids so long as the additional nucleotides or amino acids do not materially alter the function of the recited sequences. The term “materially alter,” as applied to a nucleotide sequence or amino acid sequence of the present disclosure, refers to a decrease in the antigen binding activity of the encoded polypeptide or polypeptide sequence of at least 25%. For example, additional nucleotides or amino acids added to a nucleotide or an amino acid sequence of the present disclosure may be deemed to “materially alter” the encoded polypeptide or polypeptide sequence, if such additions decrease the antigen-specific binding activity by at least 25%.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references herein are incorporated herein by reference in their entirety.

### EXAMPLES

The following examples are included to illustrate embodiments of the present disclosure. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Design and Testing of FOXM1-Specific TCRs

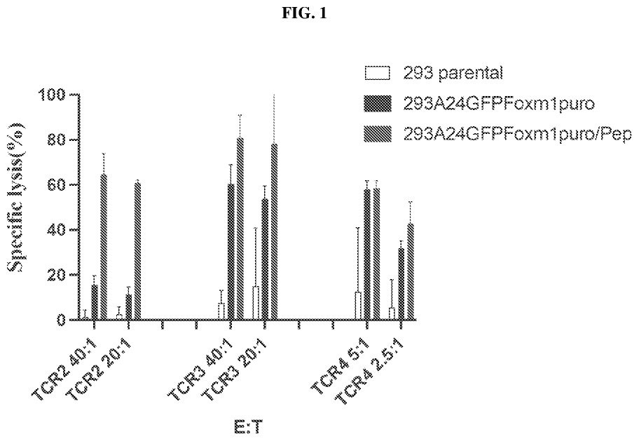

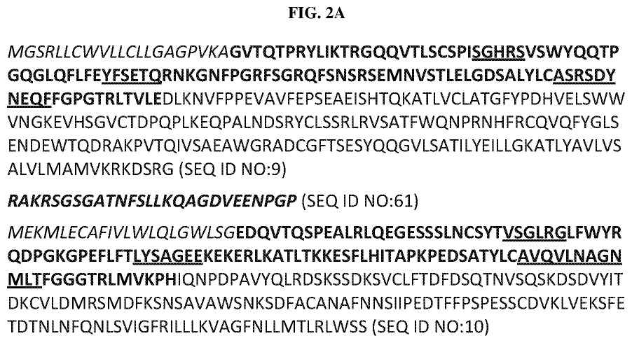

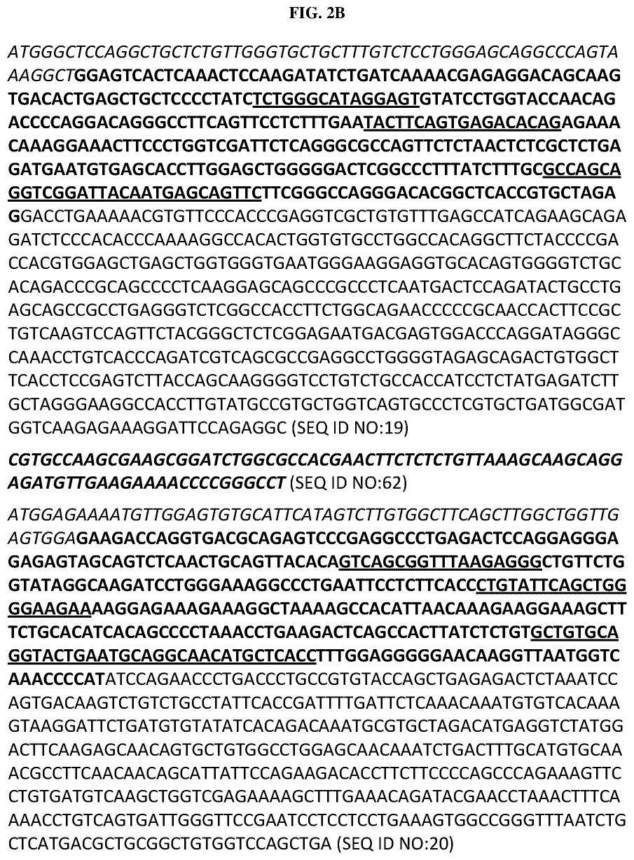

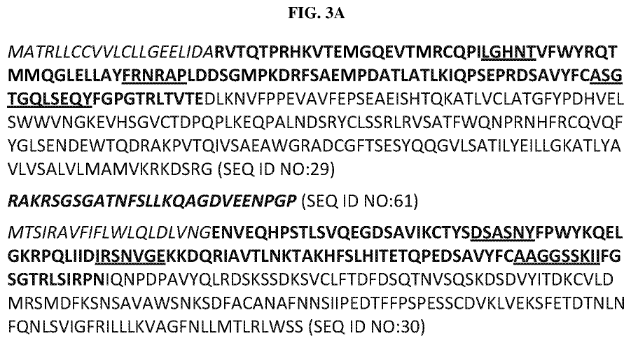

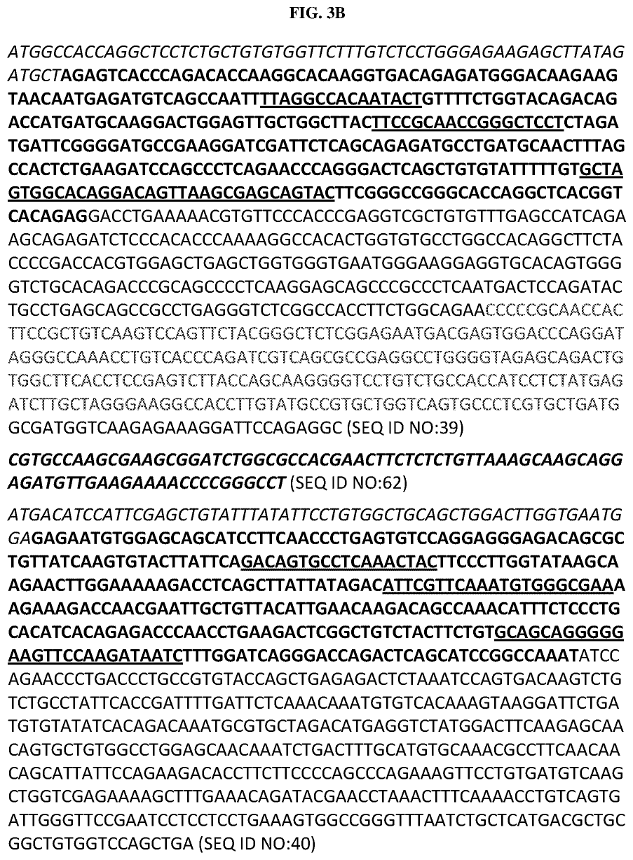

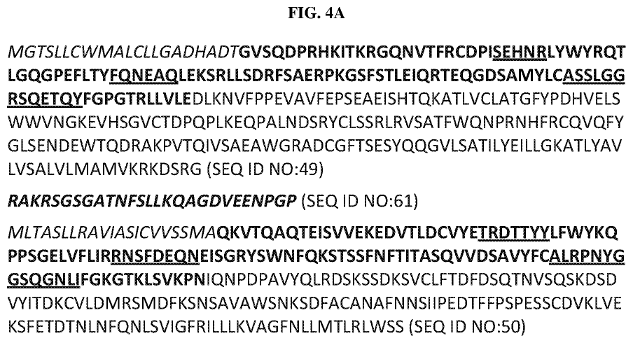

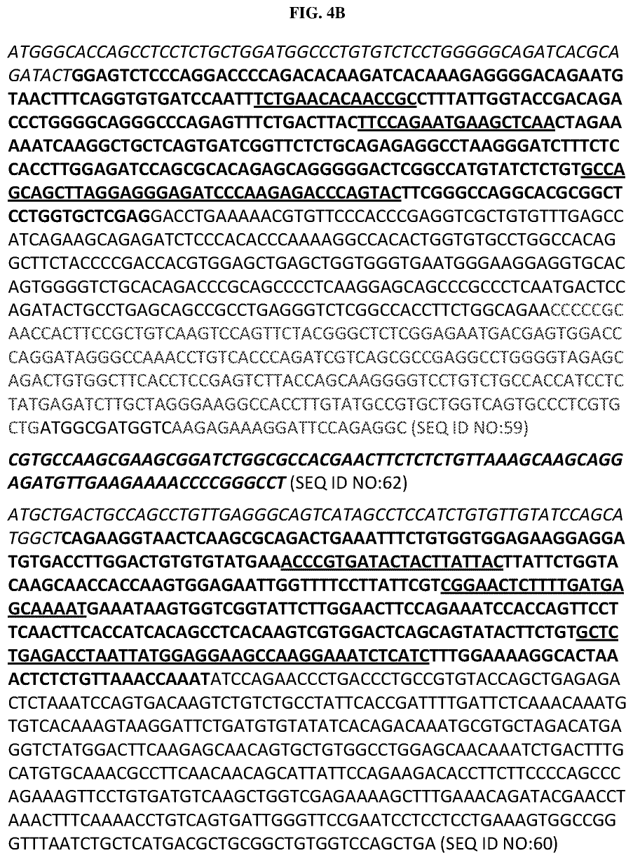

FOXM1-specific T-cells were generated from healthy donor peripheral blood mononuclear cells (PBMCs) by incubation with cells expressing an antigenic peptide derived from FOXM1 (SEQ ID NO:64) presented on HLA-A*24:02. Following stimulation with the antigenic peptide, cells were subjected to peptide/MHC tetramer-based fluorescence-activated cell sorting (FACS). Nucleotide sequences encoding the TCRα and TCRβ chains of antigen-specific tetramer-positive T-cells (TCR2, TCR3, and TCR4) were isolated, amplified, and sequenced. The nucleotide sequences encoding the TCRα and TCRβ chains of selected antigen-specific T-cell receptors (TCR2, TCR3, and TCR4) were then transduced into PBMCs. TCR-transduced PBMCs were cultured with wild-type 293 cells (white bars), 293 cells endogenously expressing FOXM1 and HLA-A*24:02 (black bars), or 293 cells endogenously expressing HLA-A*24:02 and pulsed with FOXM1 peptide (SEQ ID NO:64) (dark grey bars) (FIG. 1) at different effector cell to target cell ratios (40:1, 20:1, 5:1, and 2.5:1). As shown in FIG. 1, CD8-positive T-cells transduced with a FOXM1-specific TCR were capable of selectively recognizing and killing FOXM1/HLA-A*24:02-positive target cells. This demonstrates that the neoantigen peptide (SEQ ID NO:64) can be naturally processed and presented on HLA-A*24:02 from the full-length FOXM1 protein. FIG. 2A and FIG. 2B show the amino acid and nucleotide sequences of the validated FOXM1/HLA-A*24:02-specific T-cell receptor TCR2. FIG. 3A and FIG. 3B show the amino acid and nucleotide sequences of the validated FOXM1/HLA-A*24:02-specific T-cell receptor TCR3. FIG. 4A and FIG. 4B show the amino acid and nucleotide sequences of the validated FOXM1/HLA-A*24:02-specific T-cell receptor TCR4.

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A recombinant polynucleotide molecule comprising a nucleotide sequence that encodes an antigen binding variable region comprising a CDR3 region, wherein said CDR3 region comprises an amino acid sequence selected from the group consisting of SEQ ID NO:3, SEQ ID NO:23, and SEQ ID NO:43.

2. The recombinant polynucleotide molecule of claim 1, wherein:
a) the CDR3 region comprises the amino acid sequence of SEQ ID NO:3, and the antigen binding variable region further comprises a CDR1 region comprising the amino acid sequence of SEQ ID NO:1;
b) the CDR3 region comprises the amino acid sequence of SEQ ID NO:3, and the antigen binding variable region further comprises a CDR2 region comprising the amino acid sequence of SEQ ID NO:2;
c) the CDR3 region comprises the amino acid sequence of SEQ ID NO:23, and the antigen binding variable region further comprises a CDR1 region comprising the amino acid sequence of SEQ ID NO:21;
d) the CDR3 region comprises the amino acid sequence of SEQ ID NO:23, and the antigen binding variable region further comprises a CDR2 region comprising the amino acid sequence of SEQ ID NO:22;
e) the CDR3 region comprises the amino acid sequence of SEQ ID NO:43, and the antigen binding variable region further comprises a CDR1 region comprising the amino acid sequence of SEQ ID NO:41; or
f) the CDR3 region comprises the amino acid sequence of SEQ ID NO:43, and the antigen binding variable region further comprises a CDR2 region comprising the amino acid sequence of SEQ ID NO:42.

3. The recombinant polynucleotide molecule of claim 1, wherein:
a) the CDR3 region comprises the amino acid sequence of SEQ ID NO:3, and the antigen binding variable region further comprises an amino acid sequence having at least about 85% sequence identity to the amino acid sequence of SEQ ID NO:4;
b) the CDR3 region comprises the amino acid sequence of SEQ ID NO:23, and the antigen binding variable region further comprises an amino acid sequence having at least about 85% sequence identity to the amino acid sequence of SEQ ID NO:24; or
c) the CDR3 region comprises the amino acid sequence of SEQ ID NO:43, and the antigen binding variable region further comprises an amino acid sequence having at least about 85% sequence identity to the amino acid sequence of SEQ ID NO:44.

4. A polypeptide encoded by the recombinant polynucleotide molecule of claim 1.

5. The polypeptide of claim 4, wherein said antigen binding variable region specifically binds an antigenic peptide derived from FOXM1.

6. A composition comprising the polypeptide of claim 4.

7. A cell comprising the recombinant polynucleotide molecule of claim 1.

8. A method of producing an engineered cell, the method comprising introducing the recombinant polynucleotide molecule of claim 1 into a cell.

9. A method of treating cancer, the method comprising administering the polypeptide of claim 4 to a subject in need thereof.

10. The method of claim 9, wherein the cancer comprises a cancer cell expressing a FOXM1 protein presented on HLA-A*24:02.

11. The method of claim 10, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

12. A method of treating cancer, the method comprising administering the cell of claim 7 to a subject in need thereof.

13. The method of claim 12, wherein the cancer comprises a cancer cell expressing a FOXM1 protein presented on HLA-A*24:02.

14. The method of claim 13, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

15. A recombinant polynucleotide molecule comprising a nucleotide sequence that encodes an antigen binding variable region comprising a CDR3 region, wherein said CDR3 region comprises an amino acid sequence selected from the group consisting of SEQ ID NO:7, SEQ ID NO:27, and SEQ ID NO:47.

16. The recombinant polynucleotide molecule of claim 15, wherein:
a) the CDR3 region comprises the amino acid sequence of SEQ ID NO:7, and the antigen binding variable region further comprises a CDR1 region comprising the amino acid sequence of SEQ ID NO:5;
b) the CDR3 region comprises the amino acid sequence of SEQ ID NO:7, and the antigen binding variable region further comprises a CDR2 region comprising the amino acid sequence of SEQ ID NO:6;
c) the CDR3 region comprises the amino acid sequence of SEQ ID NO:27, and the antigen binding variable region further comprises a CDR1 region comprising the amino acid sequence of SEQ ID NO:25;
d) the CDR3 region comprises the amino acid sequence of SEQ ID NO:27, and the antigen binding variable region further comprises a CDR2 region comprising the amino acid sequence of SEQ ID NO:26;
e) the CDR3 region comprises the amino acid sequence of SEQ ID NO:47, and the antigen binding variable region further comprises a CDR1 region comprising the amino acid sequence of SEQ ID NO:45; or
f) the CDR3 region comprises the amino acid sequence of SEQ ID NO:47, and the antigen binding variable region further comprises a CDR2 region comprising the amino acid sequence of SEQ ID NO:46.

17. The recombinant polynucleotide molecule of claim 15, wherein:
a) the CDR3 region comprises the amino acid sequence of SEQ ID NO:7, and the antigen binding variable region further comprises an amino acid sequence having at least about 85% sequence identity to the amino acid sequence of SEQ ID NO:8;
b) the CDR3 region comprises the amino acid sequence of SEQ ID NO:27, and the antigen binding variable region further comprises an amino acid sequence having at least about 85% sequence identity to the amino acid sequence of SEQ ID NO:28; or
c) the CDR3 region comprises the amino acid sequence of SEQ ID NO:47, and the antigen binding variable region further comprises an amino acid sequence having at least about 85% sequence identity to the amino acid sequence of SEQ ID NO:48.

18. A polypeptide encoded by the recombinant polynucleotide molecule of claim 15.

19. The polypeptide of claim 18, wherein said antigen binding variable region specifically binds an antigenic peptide derived from FOXM1.

20. A composition comprising the polypeptide of claim 18.

21. A cell comprising the recombinant polynucleotide molecule of claim 15.

22. A method of producing an engineered cell, the method comprising introducing the recombinant polynucleotide molecule of claim 15 into a cell.

23. A method of treating cancer, the method comprising administering the polypeptide of claim 18 to a subject in need thereof.

24. The method of claim 23, wherein the cancer comprises a cancer cell expressing a FOXM1 protein presented on HLA-A*24:02.

25. The method of claim 24, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

26. A method of treating cancer, the method comprising administering the cell of claim 21 to a subject in need thereof.

27. The method of claim 26, wherein the cancer comprises a cancer cell expressing a FOXM1 protein presented on HLA-A*24:02.

28. The method of claim 27, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

29. A composition comprising a polypeptide, wherein said polypeptide comprises an antigen binding variable region comprising a CDR3 region, wherein said CDR3 region comprises an amino acid sequence selected from the group consisting of SEQ ID NO:3, SEQ ID NO:7, SEQ ID NO:23, SEQ ID NO:27, SEQ ID NO:43, and SEQ ID NO:47.

30. The composition of claim 29, wherein:
a) the CDR3 region comprises the amino acid sequence of SEQ ID NO:3, and the antigen binding variable region further comprises a CDR1 region comprising the amino acid sequence of SEQ ID NO:1;
b) the CDR3 region comprises the amino acid sequence of SEQ ID NO:3, and the antigen binding variable region further comprises a CDR2 region comprising the amino acid sequence of SEQ ID NO:2;
c) the CDR3 region comprises the amino acid sequence of SEQ ID NO:7, and the antigen binding variable region further comprises a CDR1 region comprising the amino acid sequence of SEQ ID NO:5;
d) the CDR3 region comprises the amino acid sequence of SEQ ID NO:7, and the antigen binding variable region further comprises a CDR2 region comprising the amino acid sequence of SEQ ID NO:6;
e) the CDR3 region comprises the amino acid sequence of SEQ ID NO:23, and the antigen binding variable region further comprises a CDR1 region comprising the amino acid sequence of SEQ ID NO:21;
f) the CDR3 region comprises the amino acid sequence of SEQ ID NO:23, and the antigen binding variable region further comprises a CDR2 region comprising the amino acid sequence of SEQ ID NO:22;
g) the CDR3 region comprises the amino acid sequence of SEQ ID NO:27, and the antigen binding variable region further comprises a CDR1 region comprising the amino acid sequence of SEQ ID NO:25;
h) the CDR3 region comprises the amino acid sequence of SEQ ID NO:27, and the antigen binding variable region further comprises a CDR2 region comprising the amino acid sequence of SEQ ID NO:26;
i) the CDR3 region comprises the amino acid sequence of SEQ ID NO:43, and the antigen binding variable region further comprises a CDR1 region comprising the amino acid sequence of SEQ ID NO:41;
j) the CDR3 region comprises the amino acid sequence of SEQ ID NO:43, and the antigen binding variable region further comprises a CDR2 region comprising the amino acid sequence of SEQ ID NO:42;
k) the CDR3 region comprises the amino acid sequence of SEQ ID NO:47, and the antigen binding variable region further comprises a CDR1 region comprising the amino acid sequence of SEQ ID NO:45;
l) the CDR3 region comprises the amino acid sequence of SEQ ID NO:47, and the antigen binding variable region further comprises a CDR2 region comprising the amino acid sequence of SEQ ID NO:46;
m) the CDR3 region comprises the amino acid sequence of SEQ ID NO:3, and the antigen binding variable region further comprise an amino acid sequence having at least about 85% sequence identity to the amino acid sequence of SEQ ID NO:4;
n) the CDR3 region comprises the amino acid sequence of SEQ ID NO:7, and the antigen binding variable region further comprise an amino acid sequence having at least about 85% sequence identity to the amino acid sequence of SEQ ID NO:8;
o) the CDR3 region comprises the amino acid sequence of SEQ ID NO:23, and the antigen binding variable region further comprise an amino acid sequence having at least about 85% sequence identity to the amino acid sequence of SEQ ID NO:24;
p) the CDR3 region comprises the amino acid sequence of SEQ ID NO:27, and the antigen binding variable region further comprise an amino acid sequence having at least about 85% sequence identity to the amino acid sequence of SEQ ID NO:28;
q) the CDR3 region comprises the amino acid sequence of SEQ ID NO:43, and the antigen binding variable region further comprise an amino acid sequence having at least about 85% sequence identity to the amino acid sequence of SEQ ID NO:44; or
r) the CDR3 region comprises the amino acid sequence of SEQ ID NO:47, and the antigen binding variable region further comprise an amino acid sequence having at least about 85% sequence identity to the amino acid sequence of SEQ ID NO:48.

31. The composition of claim 29, wherein said composition is serum-free, endotoxin-free, or sterile.

32. A method of treating cancer, the method comprising administering the composition of claim 29 to a subject in need thereof.

33. The method of claim 32, wherein the cancer comprises a cancer cell expressing a FOXM1 protein presented on HLA-A*24:02.

34. The method of claim 33, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

35. A method for treating cancer, the method comprising administering a population of activated T-cells that comprise a polypeptide to a subject in need thereof, wherein said polypeptide comprises an antigen binding variable region comprising a CDR3 region, and wherein said CDR3 region comprises an amino acid sequence selected from the group consisting of SEQ ID NO:3, SEQ ID NO:7, SEQ ID NO:23, SEQ ID NO:27, SEQ ID NO:43, and SEQ ID NO:47.

36. The method of claim 35, wherein the cancer comprises a cancer cell expressing a FOXM1 protein presented on HLA-A*24:02.

37. The method of claim 36, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

38. The method of claim 35, wherein:
a) the CDR3 region comprises the amino acid sequence of SEQ ID NO:3, and the antigen binding variable region further comprises a CDR1 region comprising the amino acid sequence of SEQ ID NO:1;
b) the CDR3 region comprises the amino acid sequence of SEQ ID NO:3, and the antigen binding variable region further comprises a CDR2 region comprising the amino acid sequence of SEQ ID NO:2;
c) the CDR3 region comprises the amino acid sequence of SEQ ID NO:7, and the antigen binding variable region further comprises a CDR1 region comprising the amino acid sequence of SEQ ID NO:5;
d) the CDR3 region comprises the amino acid sequence of SEQ ID NO:7, and the antigen binding variable region further comprises a CDR2 region comprising the amino acid sequence of SEQ ID NO:6;
e) the CDR3 region comprises the amino acid sequence of SEQ ID NO:23, and the antigen binding variable region further comprises a CDR1 region comprising the amino acid sequence of SEQ ID NO:21;
f) the CDR3 region comprises the amino acid sequence of SEQ ID NO:23, and the antigen binding variable region further comprises a CDR2 region comprising the amino acid sequence of SEQ ID NO:22;
g) the CDR3 region comprises the amino acid sequence of SEQ ID NO:27, and the antigen binding variable region further comprises a CDR1 region comprising the amino acid sequence of SEQ ID NO:25;
h) the CDR3 region comprises the amino acid sequence of SEQ ID NO:27, and the antigen binding variable region further comprises a CDR2 region comprising the amino acid sequence of SEQ ID NO:26;
i) the CDR3 region comprises the amino acid sequence of SEQ ID NO:43, and the antigen binding variable region further comprises a CDR1 region comprising the amino acid sequence of SEQ ID NO:41;
j) the CDR3 region comprises the amino acid sequence of SEQ ID NO:43, and the antigen binding variable region further comprises a CDR2 region comprising the amino acid sequence of SEQ ID NO:42;
k) the CDR3 region comprises the amino acid sequence of SEQ ID NO:47, and the antigen binding variable region further comprises a CDR1 region comprising the amino acid sequence of SEQ ID NO:45;
l) the CDR3 region comprises the amino acid sequence of SEQ ID NO:47, and the antigen binding variable region further comprises a CDR2 region comprising the amino acid sequence of SEQ ID NO:46;
m) the CDR3 region comprises the amino acid sequence of SEQ ID NO:3, and the antigen binding variable region further comprise an amino acid sequence having at least about 85% sequence identity to the amino acid sequence of SEQ ID NO:4;
n) the CDR3 region comprises the amino acid sequence of SEQ ID NO:7, and the antigen binding variable region further comprise an amino acid sequence having at least about 85% sequence identity to the amino acid sequence of SEQ ID NO:8;
o) the CDR3 region comprises the amino acid sequence of SEQ ID NO:23, and the antigen binding variable region further comprise an amino acid sequence having at least about 85% sequence identity to the amino acid sequence of SEQ ID NO:24;
p) the CDR3 region comprises the amino acid sequence of SEQ ID NO:27, and the antigen binding variable region further comprise an amino acid sequence having at least about 85% sequence identity to the amino acid sequence of SEQ ID NO:28;
q) the CDR3 region comprises the amino acid sequence of SEQ ID NO:43, and the antigen binding variable region further comprise an amino acid sequence having at least about 85% sequence identity to the amino acid sequence of SEQ ID NO:44; or
r) the CDR3 region comprises the amino acid sequence of SEQ ID NO:47, and the antigen binding variable region further comprise an amino acid sequence having at least about 85% sequence identity to the amino acid sequence of SEQ ID NO:48.

39. A recombinant T-cell receptor comprising a TCRβ chain variable region and a TCRα chain variable region, wherein:
a) the TCRβ chain variable region comprises a CDR3 region comprising the amino acid sequence of SEQ ID NO:3, and further comprises an amino acid sequence having at least about 85%, about 90%, or about 95% sequence identity to the amino acid sequence of SEQ ID NO:4, and wherein the TCRα chain variable region comprises a CDR3 region comprising the amino acid sequence of SEQ ID NO:7, and further comprises an amino acid sequence having at least about 85%, about 90%, or about 95% sequence identity to the amino acid sequence of SEQ ID NO:8;
b) the TCRβ chain variable region comprises a CDR3 region comprising the amino acid sequence of SEQ ID NO:23, and further comprises an amino acid sequence having at least about 85%, about 90%, or about 95% sequence identity to the amino acid sequence of SEQ ID NO:24, and wherein the TCRα chain variable region comprises a CDR3 region comprising the amino acid sequence of SEQ ID NO:27, and further comprises an amino acid sequence having at least about 85%, about 90%, or about 95% sequence identity to the amino acid sequence of SEQ ID NO:28; or
c) the TCRβ chain variable region comprises a CDR3 region comprising the amino acid sequence of SEQ ID NO:43, and further comprises an amino acid sequence having at least about 85%, about 90%, or about 95% sequence identity to the amino acid sequence of SEQ ID NO:44, and wherein the TCRα chain variable region comprises a CDR3 region comprising the amino acid sequence of SEQ ID NO:47, and further comprises an amino acid sequence having at least about 85%, about 90%, or about 95% sequence identity to the amino acid sequence of SEQ ID NO:48.

40. The recombinant T-cell receptor of claim 39, wherein:
a) the TCRβ chain variable region comprises the amino acid sequence of SEQ ID NO: 4, and wherein the TCRα chain variable region comprises the amino acid sequence SEQ ID NO:8;
b) the TCRβ chain variable region comprises the amino acid sequence of SEQ ID NO: 24, and wherein the TCRα chain variable region comprises the amino acid sequence SEQ ID NO:28; or
c) the TCRβ chain variable region comprises the amino acid sequence of SEQ ID NO: 44, and wherein the TCRα chain variable region comprises the amino acid sequence SEQ ID NO:48.

41. The recombinant T-cell receptor of claim 39, wherein the recombinant T-cell receptor is a single chain T-cell receptor.

42. A cell comprising the recombinant T-cell receptor of claim 39.

43. A cell comprising the recombinant T-cell receptor of claim 40.

44. A cell comprising the recombinant T-cell receptor of claim 41.

45. A composition comprising a T-cell receptor comprising a TCRβ chain variable region and a TCRα chain variable region, wherein:
a) the TCRβ chain variable region comprises a CDR3 region comprising the amino acid sequence of SEQ ID NO:3, and further comprises an amino acid sequence having at least about 85%, about 90%, or about 95% sequence identity to the amino acid sequence of SEQ ID NO:4, and wherein the TCRα chain variable region comprises a CDR3 region comprising the amino acid sequence of SEQ ID NO:7, and further comprises an amino acid sequence having at least about 85%, about 90%, or about 95% sequence identity to the amino acid sequence of SEQ ID NO:8;
b) the TCRβ chain variable region comprises a CDR3 region comprising the amino acid sequence of SEQ ID NO:23, and further comprises an amino acid sequence having at least about 85%, about 90%, or about 95% sequence identity to the amino acid sequence of SEQ ID NO:24, and wherein the TCRα chain variable region comprises a CDR3 region comprising the amino acid sequence of SEQ ID NO:27, and further comprises an amino acid sequence having at least about 85%, about 90%, or about 95% sequence identity to the amino acid sequence of SEQ ID NO:28; or
c) the TCRβ chain variable region comprises a CDR3 region comprising the amino acid sequence of SEQ ID NO:43, and further comprises an amino acid sequence having at least about 85%, about 90%, or about 95% sequence identity to the amino acid sequence of SEQ ID NO:44, and wherein the TCRα chain variable region comprises a CDR3 region comprising the amino acid sequence of SEQ ID NO:47, and further comprises an amino acid sequence having at least about 85%, about 90%, or about 95% sequence identity to the amino acid sequence of SEQ ID NO:48.

46. The composition of claim 45, wherein:
a) the TCRβ chain variable region comprises the amino acid sequence of SEQ ID NO: 4, and wherein the TCRα chain variable region comprises the amino acid sequence SEQ ID NO:8;
b) the TCRβ chain variable region comprises the amino acid sequence of SEQ ID NO: 24, and wherein the TCRα chain variable region comprises the amino acid sequence SEQ ID NO:28; or
c) the TCRβ chain variable region comprises the amino acid sequence of SEQ ID NO: 44, and wherein the TCRα chain variable region comprises the amino acid sequence SEQ ID NO:48.

47. The composition of claim 45, wherein the T-cell receptor is a single chain T-cell receptor.

48. The composition of claim 45, wherein the composition comprises a plurality of cells comprising said T-cell receptor.

49. The composition of claim 46, wherein the composition comprises a plurality of cells comprising said T-cell receptor.

50. The composition of claim 47, wherein the composition comprises a plurality of cells comprising said T-cell receptor.

51. A method of treating cancer, the method comprising administering the recombinant T-cell receptor of claim 39 to a subject in need thereof.

52. The method of claim 51, wherein the cancer comprises a cancer cell expressing a FOXM1 protein presented on HLA-A*24:02.

53. The method of claim 52, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

54. A method of treating cancer, the method comprising administering the cell of claim 42 to a subject in need thereof.

55. The method of claim 54, wherein the cancer comprises a cancer cell expressing a FOXM1 protein presented on HLA-A*24:02.

56. The method of claim 55, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

57. A method of treating cancer, the method comprising administering the composition of claim 45 to a subject in need thereof.

58. The method of claim 57, wherein the cancer comprises a cancer cell expressing a FOXM1 protein presented on HLA-A*24:02.

59. The method of claim 58, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

60. A bi-specific T-cell engager comprising a TCRβ chain variable region and a TCRα chain variable region, wherein:
a) the TCRβ chain variable region comprises a CDR3 region comprising the amino acid sequence of SEQ ID NO:3, and wherein the TCRα chain variable region comprises a CDR3 region comprising the amino acid sequence of SEQ ID NO:7;
b) the TCRβ chain variable region comprises a CDR3 region comprising the amino acid sequence of SEQ ID NO:23, and wherein the TCRα chain variable region comprises a CDR3 region comprising the amino acid sequence of SEQ ID NO:27; or
c) the TCRβ chain variable region comprises a CDR3 region comprising the amino acid sequence of SEQ ID NO:43, and wherein the TCRα chain variable region comprises a CDR3 region comprising the amino acid sequence of SEQ ID NO:47.

61. The bi-specific T-cell engager of claim 60, wherein:
a) the CDR3 region comprises the amino acid sequence of SEQ ID NO:3, and the antigen binding variable region further comprises a CDR1 region comprising the amino acid sequence of SEQ ID NO:1;
b) the CDR3 region comprises the amino acid sequence of SEQ ID NO:3, and the antigen binding variable region further comprises a CDR2 region comprising the amino acid sequence of SEQ ID NO:2;
c) the CDR3 region comprises the amino acid sequence of SEQ ID NO:7, and the antigen binding variable region further comprises a CDR1 region comprising the amino acid sequence of SEQ ID NO:5;
d) the CDR3 region comprises the amino acid sequence of SEQ ID NO:7, and the antigen binding variable region further comprises a CDR2 region comprising the amino acid sequence of SEQ ID NO:6;
e) the CDR3 region comprises the amino acid sequence of SEQ ID NO:23, and the antigen binding variable region further comprises a CDR1 region comprising the amino acid sequence of SEQ ID NO:21;
f) the CDR3 region comprises the amino acid sequence of SEQ ID NO:23, and the antigen binding variable region further comprises a CDR2 region comprising the amino acid sequence of SEQ ID NO:22;
g) the CDR3 region comprises the amino acid sequence of SEQ ID NO:27, and the antigen binding variable region further comprises a CDR1 region comprising the amino acid sequence of SEQ ID NO:25;
h) the CDR3 region comprises the amino acid sequence of SEQ ID NO:27, and the antigen binding variable region further comprises a CDR2 region comprising the amino acid sequence of SEQ ID NO:26;
i) the CDR3 region comprises the amino acid sequence of SEQ ID NO:43, and the antigen binding variable region further comprises a CDR1 region comprising the amino acid sequence of SEQ ID NO:41;
j) the CDR3 region comprises the amino acid sequence of SEQ ID NO:43, and the antigen binding variable region further comprises a CDR2 region comprising the amino acid sequence of SEQ ID NO:42;
k) the CDR3 region comprises the amino acid sequence of SEQ ID NO:47, and the antigen binding variable region further comprises a CDR1 region comprising the amino acid sequence of SEQ ID NO:45;
l) the CDR3 region comprises the amino acid sequence of SEQ ID NO:47, and the antigen binding variable region further comprises a CDR2 region comprising the amino acid sequence of SEQ ID NO:46;
m) the CDR3 region comprises the amino acid sequence of SEQ ID NO:3, and the antigen binding variable region further comprise an amino acid sequence having at least about 85% sequence identity to the amino acid sequence of SEQ ID NO:4;
n) the CDR3 region comprises the amino acid sequence of SEQ ID NO:7, and the antigen binding variable region further comprise an amino acid sequence having at least about 85% sequence identity to the amino acid sequence of SEQ ID NO:8;
o) the CDR3 region comprises the amino acid sequence of SEQ ID NO:23, and the antigen binding variable region further comprise an amino acid sequence having at least about 85% sequence identity to the amino acid sequence of SEQ ID NO:24;
p) the CDR3 region comprises the amino acid sequence of SEQ ID NO:27, and the antigen binding variable region further comprise an amino acid sequence having at least about 85% sequence identity to the amino acid sequence of SEQ ID NO:28;
q) the CDR3 region comprises the amino acid sequence of SEQ ID NO:43, and the antigen binding variable region further comprise an amino acid sequence having at least about 85% sequence identity to the amino acid sequence of SEQ ID NO:44; or
r) the CDR3 region comprises the amino acid sequence of SEQ ID NO:47, and the antigen binding variable region further comprise an amino acid sequence having at least about 85% sequence identity to the amino acid sequence of SEQ ID NO:48.

62. A cell comprising the bi-specific T-cell engager of claim 60.

63. A composition comprising the bi-specific T-cell engager of claim 60.

64. A method of treating cancer, the method comprising administering the bi-specific T-cell engager of claim 60 to a subject in need thereof.

65. The method of claim 64, wherein the cancer comprises a cancer cell expressing a FOXM1 protein presented on HLA-A*24:02.

66. The method of claim 65, wherein the cancer cell is selected from the group consisting of a lung cancer cell, a non-small cell lung cancer (NSCLC) cell, a colorectal cancer cell, a pancreatic cancer cell, an appendiceal cancer cell, a small bowel adenocarcinoma cell, a hepatobiliary cancer cell, a gynecological malignancy cell, a hematopoietic cancer cell, a breast cancer cell, a bladder cancer cell, a prostate cancer cell, and a skin cancer cell.

67. A chimeric antigen receptor comprising the polypeptide of claim 4.

68. A chimeric antigen receptor comprising the polypeptide of claim 18.

69. A cell comprising the chimeric antigen receptor of claim 67.

70. A cell comprising the chimeric antigen receptor of claim 68.
